(19) United States
(12) Patent Application Publication
(10) Pub. No.: US 2024/0325565 A1
(43) Pub. Date: Oct. 3, 2024

(54) COMBINATION TREATMENT FOR SOLID TUMORS INCLUDING A STING AGONIST AND A CCR2 ANTAGONIST

(71) Applicant: University of Pittsburgh - Of the Commonwealth System of Higher Education, Pittsburgh, PA (US)

(72) Inventors: Song Li, Mars, PA (US); Jingjing Sun, Elkhorn, NE (US)

(21) Appl. No.: 18/624,989

(22) Filed: Apr. 2, 2024

Related U.S. Application Data
(60) Provisional application No. 63/456,654, filed on Apr. 3, 2023.

(51) Int. Cl. A61K 47/69; A61K 39/395; A61K 47/58; A61K 47/60

(52) U.S. Cl. CPC A61K 47/6925; A61K 39/3955; A61K 47/58; A61K 47/60; B82Y 5/00

(57) ABSTRACT

A formulation including a nanostructure including an agent to activate a STING pathway in vivo and a C-C chemokine receptor type 2 (CCR2) antagonist.

### CROSS-REFERENCE TO RELATED APPLICATIONS

This application claims benefit of U.S. Provisional Patent Application Ser. No. 63/456,654, filed Apr. 3, 2023, the disclosure of which is incorporated herein by reference.

### GOVERNMENTAL INTEREST

This invention was made with government support under grant numbers CA249649 and CA219399 awarded by the National Institutes of Health. The government has certain rights in the invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Pancreatic ductal adenocarcinoma (PDAC) has the lowest 5-year-survival rate (˜8%) among cancers. The lack of effective therapeutic strategy for PDAC is mainly attributed to its unique tumor microenvironment. The dense desmoplastic stroma not only restricts penetration of nanomedicine to the core of tumors, but also affects the infiltration of effector T cells, leading to an immunologically tolerant environment and driving treatment resistance. Despite the success of immune checkpoint blockade (ICB) therapy in immunogenically “hot” tumors, such therapy is less effective in treating immunogenically “cold” tumors such as PDAC. Recently, activation of the innate immune system has been demonstrated to be a promising strategy to re-engage immune cold tumors to initiate anti-cancer immunity.

Stimulator of interferon genes (STING), an endoplasmic reticulum transmembrane protein, plays an important role in innate immunity against cancer. STING is activated when the cytosolic DNA (both exogenous and endogenous) is detected and processed by cyclic guanosine monophosphate-adenosine monophosphate synthase (cGAS). STING activation triggers downstream signaling cascades, such as recruitment and phosphorylation of TANK-binding kinase-1 (TBK1) and interferon regulatory factor (IRF3), resulting in the production of type 1 interferons (1FNs), particularly IFN-β and other chemokines, which promotes the activation of antigen presenting cells (APC) as well as the priming and infiltration of T-cell to tumor sites. Tremendous efforts have been made in developing STING agonists for cancer immunotherapy. STING agonists include, for example, cyclic dinucleotide (CDN)-based agents and non CDN-based small molecules (for example, DMXAA (dimethylxanthone acetic acid), G10 (4-[(2-chloro-6-fluorophenyl)methyl]-N-(furan-2-ylmethyl)-3-oxo-1,4-benzothiazine-6-carboxamide) and α-Mangostin (1,3,6-trihydroxy-7-methoxy-2,8-bis(3-methylbut-2-en-1-yl)-9H-xanthen-9-one)). Recent studies also demonstrated that some chemotherapeutic agents, such as platinum drugs, doxorubicin and low-dose camptothecin were able to activate the STING pathway via inflicting DNA double-strand breaks (DSBs).

However, most STING agonists need to be administered intratumorally because of their metabolic instability and poor pharmacokinetic properties, which limited their clinical applications. To address this issue, efforts are being made to develop STING agonists of improved physiochemical property and/or suitable formulations for systemic administration. Nevertheless, increasing evidence has shown that the STING activation in tumor immunity is multi-faceted. Despite that Type I interferons and STING-inducible cytokines play important roles in directing dendritic cells (DC) function and T cell priming to initiate anti-tumor response, overproduction of certain cytokines was found to result in immune resistance as well as tissue damage and pathological inflammation. For example, it has been reported that aberrant packaging of the mitochondrial DNA (mtDNA) could activate cGAS-STING innate immune pathway, resulting in IFN production and cytokine release, which contributes significantly to cell death and kidney failure. In addition, it has been reported that radiation-induced STING activation led to increased expression of chemokines CCL2 and CCL7, which played an important role in tumor radio-resistance. However, current investigations focus mainly on the strategies to activate STING, developing an effective strategy to simultaneously activate STING while counteracting the overproduction of pro-immune resistance chemokines has rarely been reported.

It remains desirable to develop improved treatments for PDAC and other solid tumor cancers.

### SUMMARY

In one aspect, a formulation includes a nanostructure including an agent to activate a STING pathway in vivo and a C-C chemokine receptor type 2 (CCR2) antagonist. The agent to activate a STING pathway in vivo may include amphiphilic polymers which self-assemble into the nanostructure (for example, a micelle). The nanostructures further include a C-C chemokine receptor type 2 (CCR2) antagonist incorporated in the nanostructures.

The nanostructures may, for example, have a diameter no greater than 100 nm, a diameter no greater than 50 nm, no greater than 30 nm, or no greater than 20 nm. The nanostructures may desirably have a diameter no greater than 30 nm. Nanostructures having a diameter no greater than 30 nm are sometimes referred to herein as ultra-small nanostructures. In a number of embodiments, the micelles have a diameter in the range of 10 to 30 nm or in the range of 10 to 20 nm. The nanostructures in a number of embodiments hereof are micelles formed from a plurality of amphiphilic polymers hereof.

In a number of embodiments, the CCR2 antagonist is incorporated in the nanostructures via conjugation to the amphiphilic polymers. In a number of embodiments, the CCR2 antagonist is incorporated in the nanostructures via (physical) loading of the CCR2 antagonist into the amphiphilic polymers.

In a number of embodiments, the amphiphilic polymers comprise one or more molecules that are active as STING agonists conjugated thereto. The molecules that are sting agonists may be selected from the group consisting of nucleoside analogs and nucleotide analogs. In a number of embodiments in which the STING agonist is a nucleotide analog, the nucleotide analog is a cyclic nucleotide analog which is active as a STING agonist.

In a number of embodiment, the STING agonist is a nucleoside analog. The nucleoside analog may, for example, be a cytidine analog which is active as a STING agonist. The cytidine analog may, for example, be selected from the group consisting of gemcitabine, azacitidine, decitabine, zebularine and cytarabine (or a derivative of gemcitabine, azacitidine, decitabine, zebularine or cytarabine which is active as a STING agonist). In a number of embodiments, the STING agonist is gemcitabine (or a derivative of gemcitabine which is active as a STING agonist). When conjugated in an amphiphilic polymer hereof which self-assemble into nanostructures, nucleoside analogs may, for example, be ultra-small nanostructures.

In a number of embodiments, the one or more STING agonists are attached to the amphiphilic polymer via a linking moiety that is labile in vivo. The linking moiety that is labile in vivo may, for example, include at least one of a reductive sensitive linkage, a pH-sensitive linkage, a ROS-sensitive linkage, or a protease-sensitive linkage. In a number of embodiments, the linking moiety that is labile in vivo includes at least one of an ester bond, an orthoester bond, a thioether-ester bond, an anhydride bond, an amid bond, a carbonate bond, a disulfide bond, a hydrazone bond, a cic-acotinyl bond, an acetal bond, a carboxydimethyl maleate bond, an imine bond, an oxime bond, a silyl ether bond, a ketal bond, a thioketal bond or a protease cleavable peptide.

The CCR2 antagonist may, for example, be a small molecule compound. As used herein, the term “small molecule compound” refers to a compound having a molecular weight below 1.5 kDa. The CCR2 antagonist may, for example, have a molecular weight below 1.5 kDa. In a number of embodiments, the CCR2 antagonist is selected from the group consisting of PF-04136309, PF-04634817, BMS 813160, BMS-687681, BMS-741672, Bindarit, CCX872, CCX140, RDC018, DMX-200, AZD2423, Cenicriviroc, CNTX-6970, JNJ-17166864, MK-0812, RAP-103, RO5234444, SSR150106, WXSH0213, and NOX-E36. In a number of embodiments, the CCR2 antagonist is PF-04136309.

In a number of embodiments, the amphiphilic polymers hereof include a hydrophobic polymer backbone to which a plurality of groups are attached as pendant groups. The pendant groups include one or more molecules that are STING agonists conjugated thereto. Another plurality of pendant groups are attached to the hydrophobic polymer backbone as pendant groups and include at least one hydrophilic polymer. The hydrophobic polymer backbone may, for example, be formed via radical polymerization of vinyl monomers. The hydrophobic polymer backbone may be formed via a free radical polymerization. The hydrophobic polymer backbone may be formed via a reversible-deactivation radical polymerization.

In another aspect, a method of delivering a CCR2 antagonist includes administering a formulation as set forth above and elsewhere herein. In that regard, the formulation may include a nanostructure including an agent to activate a STING pathway in vivo and a CCR2 antagonist. The agent to activate a STING pathway in vivo may include amphiphilic polymers which self-assemble into the nanostructure (for example, a micelle). The nanostructures further include a CCR2 antagonist incorporated in the nanostructures.

As described above, the nanostructures may, for example, have a diameter no greater than 100 nm, a diameter no greater than 50 nm, no greater than 30 nm, or no greater than 20 nm. The nanostructures may desirably have a diameter no greater than 30 nm. In a number of embodiments, the micelles have a diameter in the range of 10 to 30 nm or in the range of 10 to 20 nm.

In a number of embodiments, the CCR2 antagonist is incorporated in the nanostructures via conjugation to the amphiphilic polymers. In a number of embodiments, the CCR2 antagonist is incorporated in the nanostructures via (physical) loading of the CCRT antagonist into the amphiphilic polymers.

In a number of embodiments, the amphiphilic polymers comprise one or more molecules that are active as STING agonists conjugated thereto. In a number of embodiments, the molecules that are sting agonists may, for example, be selected from the group consisting of nucleoside analogs and nucleotide analogs. As described above, a nucleotide analog may, for example, be a cyclic nucleotide analog which is active as a STING agonist. A nucleoside analog may, for example, be a cytidine analog which is active as a STING agonist. The cytidine analog may, for example, be selected from the group consisting of gemcitabine, azacitidine, decitabine, zebularine and cytarabine (or a derivative of gemcitabine, azacitidine, decitabine, zebularine or cytarabine which is active as a STING agonist). In a number of embodiments, the STING agonist is gemcitabine (or a derivative of gemcitabine which is active as a STING agonist).

In a number of embodiments, the one or more STING agonists are attached to the amphiphilic polymer via a linking moiety that is labile in vivo. The linking moiety that is labile in vivo may, for example, include at least one of a reductive sensitive linkage, a pH-sensitive linkage, a ROS-sensitive linkage, or a protease-sensitive linkage. In a number of embodiments, the linking moiety that is labile in vivo includes at least one of an ester bond, an orthoester bond, a thioether-ester bond, an anhydride bond, an amid bond, a carbonate bond, a disulfide bond, a hydrazone bond, a cic-acotinyl bond, an acetal bond, a carboxydimethyl maleate bond, an imine bond, an oxime bond, a silyl ether bond, a ketal bond, a thioketal bond or a protease cleavable peptide.

The CCR2 antagonist may, for example, be a small molecule compound. The CCR2 antagonist may, for example, have a molecular weight below 1.5 kDa. In a number of embodiments, the CCR2 antagonist is selected from the group consisting of PF-04136309, PF-04634817, BMS 813160, BMS-687681, BMS-741672, Bindarit, CCX872, CCX140, RDC018, DMX-200, AZD2423, Cenicriviroc, CNTX-6970, JNJ-17166864, MK-0812, RAP-103, RO5234444, SSR150106, WXSH0213, and NOX-E36. In a number of embodiments, the CCR2 antagonist is PF-04136309.

In a number of embodiments, the amphiphilic polymers hereof include a hydrophobic polymer backbone to which a plurality of groups are attached as pendant groups. The pendant groups include one or more molecules that are STING agonists conjugated thereto. Another plurality of pendant groups are attached to the hydrophobic polymer backbone as pendant groups and include at least one hydrophilic polymer. The hydrophobic polymer backbone may, for example, be formed via radical polymerization of vinyl monomers. The hydrophobic polymer backbone may be formed via a free radical polymerization. The hydrophobic polymer backbone may be formed via a reversible-deactivation radical polymerization.

In a number of embodiments, the method further includes co-delivering an immunotherapy agent with the formulation. The immunotherapy agent may, for example, include an anti-programmed death-i (anti PD-1) composition. The anti PD-1 composition may, for example, include a PD-1 antibody.

In another aspect, a method of formulating a composition for delivery of a first compound includes mixing amphiphilic polymers which self-assemble into nanostructures, wherein the amphiphilic polymers cause activation of a STING pathway, with a C-C chemokine receptor type 2 (CCR2) antagonist in a liquid medium.

In a further aspect, a formulation includes a nanostructure including amphiphilic polymers which self-assemble into the nanostructure. The amphiphilic polymers include one or more molecules that are STING agonists conjugated thereto. The nanostructure further includes a C-C chemokine receptor type 2 (CCR2) antagonist incorporated in the nanostructure.

In still a further aspect, a formulation includes an agent which is active to activate a STING pathway in vivo and a C-C chemokine receptor type 2 (CCR2) antagonist.

The present systems, methods, formulations and compositions along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates schematically a generalized structure of a representative embodiment of an amphiphilic polymer hereof.

FIG. 1B illustrates schematically the loading of a CCRT antagonist into a nanostructure formed by a plurality of representative amphiphilic polymer hereof.

FIG. 2A illustrates schematically another representative embodiment of the generalized structure of an amphiphilic polymer hereof.

FIG. 2B illustrates schematically another representative embodiment of the generalized structure of an amphiphilic polymer hereof including a conjugated STING agonist and a conjugated CCR2 antagonist.

FIG. 3 illustrates representative linker groups for L¹ for the polymers of FIGS. 1A, 2A and 2B.

FIG. 4 illustrates representative linker groups L² and a representative group P for the polymers of FIGS. 1A, 2A and 2B.

FIG. 5 illustrates a representative embodiment of a synthetic scheme for representative STING agonist and representative PGEM polymers hereof.

FIG. 6A Illustrates schematically the preparation of small-sized PGEM/PF micelles that effectively penetrated into PDAC solid tumors following systemic administration.

FIG. 6B illustrates schematically representative PGEM boosted STING activation and subsequent TBK1 phosphorylation and IRF3 phosphorylation, resulting in induction of IFNI) along with CCL2 and CCL7 in a proposed mechanism via which PGEM/CCR2 antagonist (PF) nanostructures such as micelles hereof exert potent anti-tumor efficacy by modulating TME and amplifying STING activation, wherein:

FIG. 6C illustrates schematically a proposed mechanism for representative PGEM-induced IFNβ expression to significantly enhance antitumor immunity through promoting DC maturation and activating both innate immunity (NK cells) and adaptive anti-tumor immunity (CD8 T cells), while both CCL2 and CCL7 help to recruit tumor associated macrophages (TAM) and MDSCs through acting on CCR2, and CCR2 antagonist PF released from PGEM/PF micelles further improves the antitumor immunity through reversing the immunosuppressive TME mediated by STING activation.

FIG. 7A illustrates the structure of three representative CCR2 antagonists, PF-04136309, BMS 813160, and Bindarit for used in the compositions hereof.

FIG. 7B illustrates the structures of several other representative CCR2 antagonists suitable for use in the compositions hereof.

FIG. 8A illustrates Western blotting for phosphorylated IRF3 (pIRF3) and total IRF3 expression as well as phosphorylated STING (pSTING) and total STING expression.

FIG. 8B illustrates normalized pIRF3 and pSTING protein levels quantified by densitometric analysis.

FIG. 8C illustrates quantification of pIRF3⁺ tumor cells after various treatments (normalized to saline treated group).

FIG. 8D illustrates quantification of pTBK1⁺ tumor cells after various treatments (normalized to saline treated group).

FIG. 8E illustrates quantification of pIRF3⁺ DCs after various treatments (normalized to saline treated group).

FIG. 8F illustrates quantification of pTBK1⁺ DCs after various treatments (normalized to saline treated group).

FIG. 8G illustrates Panc02 tumor-bearing mice were treated with saline or PGEM micelles (i.v.) plus i.p. injection with anti-IFNAR1 or the isotype control, wherein tumor sizes and body weight were measured every 4 days (n=5) and the tumor weight was obtained at the endpoint (in all figures in which asterisks are present, *P<0.05; **P<0.01; ***P<0.001; ****P<0.0001).

FIG. 9A illustrates a heat map of chemokines (CCL2, CCL7, and ISG15) expression after various treatments.

FIG. 9B illustrates the mRNA expression level of CCL2 in mouse Panc02 tumors treated with saline, GEM, and PGEM for three times.

FIG. 9C illustrates the mRNA expression level of CCL7 in mouse Panc02 tumors treated with saline, GEM, and PGEM for three times.

FIG. 9D illustrates the mRNA expression level of ISG15 in mouse Panc02 tumors treated with saline. GEM, and PGEM for three times.

FIG. 9E illustrates the mRNA expression levels of CCL2 in cultured Panc02 cell lines treated with saline, GEM, and PGEM.

FIG. 9F illustrates the mRNA expression levels of CCL7 in cultured Panc02 cell lines treated with saline, GEM, and PGEM.

FIG. 9G illustrates the mRNA expression levels of ISG15 in cultured Panc02 cell lines treated with saline, GEM, and PGEM.

FIG. 9H illustrates knock down of STING protein in Panc02 cells assessed by Western blotting after treatments with control siRNA (siNC or siSTING for 24h or 48h.

FIG. 9I illustrates the mRNA expression levels of CCL2, CCL7 and ISG15 after treatments with PGEM plus either siNC or siSTING.

FIG. 9J illustrates the mRNA expression levels of CCL2. CCL7 and ISG15 after treatments with PGEM plus either isotype control or IFNAR1 antibody.

FIG. 10A illustrates the 32-residue PGEM polymer consisted of four types of residues including PG (panel a), NPG (panel b), EM (panel c), and CEM (panel d).

FIG. 10B illustrates the time courses of radius of gyration of polymer/8-drug systems in explicit water MD simulations, wherein, for all the three drugs, RoG reached equilibria after 25 nanoseconds.

FIG. 10C illustrates the tumor growth curves after treatments of various CCL2/CCR2 inhibitors formulated in PGEM micelles.

FIG. 11A illustrates tumor growth curve after application of a combination of GEM and PF.

FIG. 11B illustrates tumor growth curve (volume) after various treatments, wherein tumors were characterized at the completion of the experiment.

FIG. 11C illustrates tumor growth curve (weight) after various treatments, wherein tumors were characterized at the completion of the experiment.

FIG. 11D illustrates photographs of H&E staining after various treatments (Scale bar: 20 μm).

FIG. 11E illustrates a safety profile of PGEM/PF micelles in which body weight is measured over time.

FIG. 11F illustrates a safety profile of PGEM/PF micelles in which kidney and liver function is determined.

FIG. 11G illustrates a safety profile of PGEM/PF micelles in which white blood cells (WBCs) are determined in peripheral blood.

FIG. 11H illustrates a safety profile of PGEM/PF micelles in which red blood cells (RBCs) are determined in peripheral blood.

FIG. 11I illustrates a safety profile of PGEM/PF micelles in which hemoglobin concentrations are determined in peripheral blood.

FIG. 11J illustrates a safety profile of PGEM/PF micelles in which in which lymphocytes are determined in peripheral blood.

FIG. 11K illustrates a safety profile of PGEM/PF micelles in which wherein monocytes are determined in peripheral blood.

FIG. 11L illustrates that PGEM/PF micelles suppressed lung metastasis of pancreatic cancer in vivo in representative lung images following various treatments.

FIG. 11M illustrates that PGEM/PF micelles suppressed lung metastasis of pancreatic cancer in vivo via the number of tumor nodules in lung tissues following various treatments.

FIG. 11N illustrates that PGEM/PF micelles suppressed lung metastasis of pancreatic cancer in vivo via H&E staining of lung tissues at the endpoint following various treatments.

FIG. 11O illustrates that PGEM/PF micelles suppressed lung metastasis of pancreatic cancer in vivo via quantification of tumor areas of H&E images following various treatments.

FIG. 12A illustrates that PGEM/PF-6309 micelles exhibited anti-tumor efficacy by activating innate and adaptive immunity in immune profiling after various treatments, wherein percentages of Myeloid Derived Suppressive Cells (MDSCs) are set forth.

FIG. 12B illustrates Tumor Associated Macrophages (TAMs) after various treatments.

FIG. 12C illustrates M2/M1 ratios after various treatments.

FIG. 12D illustrates percentages of natural killer (NK) cells after various treatments

FIG. 12E illustrates GZB producing NK cells after various treatments.

FIG. 12F illustrates IFN-γ producing NK cells after various treatments.

FIG. 12G illustrates percentages of CD8⁺ T cells after various treatments.

FIG. 12H illustrates GZB producing CD8⁺ T cells after various treatments, wherein percentages are normalized to control group.

FIG. 12I illustrates tumor volume over time wherein Panc02 tumor bearing mice were treated with saline or PGEM/PF micelles (i.v.) plus i.p. injection with anti-NK1.1, or its isotype control.

FIG. 12J illustrates tumor weight over time wherein Panc02 tumor bearing mice were treated with saline or PGEM/PF micelles (i.v.) plus i.p. injection with anti-NK1.1, or its isotype control.

FIG. 12K illustrates tumor volume over time wherein Panc02 tumor bearing mice were treated with saline or PGEM/PF micelles (i.v.) plus i.p. injection with anti-CD8, or its isotype control.

FIG. 12L illustrates tumor weight over time wherein Panc02 tumor bearing mice were treated with saline or PGEM/PF micelles (i.v.) plus i.p. injection with anti-CD8, or its isotype control, wherein tumor sizes were measured every 4 days (n=5) and the tumor weight was obtained at the endpoint in FIGS. 121 through 12L.

FIG. 13A illustrates the quantification of the percentages of PD-1⁺ cells within gated NK1.1⁺ populations in Panc02 tumors.

FIG. 13B illustrates the quantification of the percentages of PD-1 cells within gated CD8⁺ T populations in Panc02 tumors.

FIG. 13C illustrates schematically a rationale for PGEM/PF micelles in combination with anti-PD-1 regimen.

FIG. 13D illustrates the therapeutic effect of various formulations on tumor volume for Female C57BL/6 mice which were subcutaneously inoculated with equal numbers of panc02 tumor cells and received the various treatments, wherein tumor growth was followed every 4 days.

FIG. 13E illustrates the therapeutic effect of the various formulations on tumor wherein, wherein the tumors were weighed at the completion of the experiment.

FIG. 13F illustrates representative images of tumors after the completion of the experiment.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a CCR2 antagonist” includes a plurality of such CCR2 antagonists and equivalents thereof known to those skilled in the art, and so forth, and reference to “the CCR2 antagonist” is a reference to one or more such CCR2 antagonists and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

As used herein, the term “polymer” refers to a chemical compound that is made of a plurality of small molecules or monomers that are arranged in a repeating structure to form a larger molecule. Thus, a polymer is a compound having multiple repeat units (or monomer units) and includes the term “oligomer,” which is a polymer that has only a few repeat units. The term “copolymer” refers to a polymer including two or more dissimilar repeat units (including terpolymers—comprising three dissimilar repeat units—etc.). Polymers may occur naturally or be formed synthetically. The use of the term “polymer” encompasses homopolymers as well as copolymers. The term “copolymer” is used herein to include any polymer having two or more different monomers. Copolymers may, for example, include alternating copolymers, periodic copolymers, statistical copolymers, random copolymers, block copolymers, graft copolymers etc. Examples of polymers include, for example, polyalkylene oxides.

As used herein, the term “pendant” refers to a chain, group or moiety attached to a backbone chain of a long molecule such as a polymer as described above. Pendant group may be either (1) short chain or low molecular weight groups or (2) long chain or high molecular groups such as polymers. Pendant groups are sometime referred to as side groups.

As used herein, the term “nucleoside analogs” refers to structural analogs of a nucleoside. As known in the art, a nucleoside includes a nucleobase linked to a sugar. As used herein, the term “nucleotide analogs” refers to structural analog of a nucleotide. As known in the art, a nucleotide includes one to three phosphates linked to a nucleoside. The terms “cyclic nucleotide” or “cyclic nucleotide analog”, as used herein refer to compounds that include a cyclic structure with at least one nucleotide component. A cyclic nucleotide analog may, for example, include a single-phosphate with a cyclic bond arrangement between the sugar and phosphate groups. Cyclic dinucleotide analogs include cyclisation between two nucleotides via two phosphate groups thereof. In that regard, the secondary phosphate groups may be linked to each other, forming a cyclic structure with two phosphodiester bonds.

CCL2 and CCL7, which are produced by many types of cancer cells including PDAC cells, are major tumor-derived factors responsible for recruiting immunosuppressive immune cells and promoting tumor progression and metastasis. Thus, CCL2 and CCL7 may be involved in both intrinsic and acquired resistance of tumor cells to various treatments including chemotherapy. CCL2 and CCL7 share the same functional C-C chemokine receptor type 2 (CCR2), which is an attractive target for cancer therapy.

In a number of embodiments, a formulation hereof includes an agent to activate a STING pathway in vivo as well as a C-C chemokine receptor type 2 (CCR2) antagonist. The formulation is desirably a nanomedicine including nanostructure or nanoparticle including the agent to activate a STING pathway in vivo as well as the CCR2 antagonist. Various nanostructures or nanoparticles may be used for codelivery of an agent to activate a STING pathway and a CCR2 antagonist. For example, a number of formulations hereof include a nanostructure including amphiphilic polymers which self-assemble into the nanostructure.

In a number of embodiments, the amphiphilic polymers activate or cause activation of (either inherently or as a prodrug) a STING pathway in vivo. As used herein a prodrug is a pharmacological conjugate or compound that, after intake, is metabolized (that is, converted within the body) into a pharmacological drug of increased activity. The formulations further incudes a CCR2 antagonist incorporated within the nanostructures. In a number of embodiments, the CCR2 antagonist may, for example, be incorporated into nanostructures via conjugation within self-assembling amphiphilic polymers or via loading of the CCR2 antagonist into nanostructures formed by self-assembling amphiphilic polymers.

It is desirable to load or physically load the CCR2 antagonist into nanostructures hereof to, for example, facilitate formation of relatively small micelles. In general, any small molecule active as a CCR2 antagonist may be loaded into the nanostructures hereof. A review of the CCR2/CCL2 landscape is provided in Fei L, Ren X, Yu 1, Zhan Y. Targeting the CCL2/CCR2 Axis in Cancer Immunotherapy: One Stone, Three Birds? Front Immunol. 2021 Nov. 3; 12:771210. doi: 10.3389/fimmu.2021.771210. PMID: 34804061; PMCID: PMC8596464, the disclosure of which is incorporated herein by reference. As described above, in embodiments in which a CCR2 antagonist is loaded into nanostructures hereof, which are formed by self-assembling amphiphilic polymers which are functional to activate a STING pathway, a formulation hereof may, for example, be formed by mixing the amphiphilic polymers with the CCR2 antagonist in a liquid medium.

In a number of embodiments, a compound is conjugated to an amphiphilic polymer to provide activity/functionality to the amphiphilic polymer to activate a STING pathway (or, in other words, to form a STING agonist) in vivo. Such conjugation can occur during polymerization or after polymerization of the amphiphilic polymer. In a number of embodiments, the compound may, for example, be a small molecule STING agonist. In other embodiments, the compound is a small molecule that is not inherently (or measurably) considered a STING agonist, but when conjugated to the amphiphilic polymer, forms a STING agonist or has increased activity as a STING agonist. In that regard, conjugation of such a compound to an amphiphilic polymer hereof may provide increased STING activation functionality as compared to the activity of the compound. In a number of embodiments, amphiphilic compounds/polymers which are functional/active to activate a STING pathways (upon in vivo delivery to a cell) and self-assembled to from nanostructures are synthesized by conjugation of one or more molecules that are STING agonists (for example, nucleoside analogs or nucleotide analogs) to the amphiphilic compound. A nucleoside hereof may be phosphorylated inside a cell to become a nucleotide.

In a number of representative studies hereof (which may, for example, be guided by RNA sequencing (RNAseq)), it was demonstrated that representative nucleoside-conjugated, polymeric STING agonists activated the cGAS-STING innate immune pathway. In a number of such representative studies, the polymer was a gemcitabine-(GEM)-conjugated polymer (PGEM). Representative embodiments of the PGEM polymer self-assembled into ultra-small sized particles (˜13 nm) that may serve as a carrier for efficient loading of a wide variety of first molecules or drugs. The superior penetration capability of the representative small-sized PGEM carrier was confirmed in a pancreatic tumor spheroid model and an orthotopic pancreatic tumor model. However, it was also determined that the STING activation by PGEM led to increased expression of CCL2 and CCL7, resulting in tumor immune resistance. With the guidance of molecular dynamic simulations, a “one stone hits two birds” or dual-functionality formulation was created by incorporating a paired CCR2 antagonist into the representative PGEM carrier to (without limitation to any mechanism) simultaneously activate STING and overcome immune resistance, improve the overall therapeutic effect.

In a number of embodiments, the systems, formulations, methods, and compositions hereof thus provide for co-delivery of therapeutic agents or drugs (for example, therapeutic, chemotherapeutic, immunotherapeutic, etc. agents or drugs). In a number of embodiments, the formulation includes amphiphilic polymers which self-assemble into the nanostructures. A plurality of the amphiphilic polymers include one or more groups attached or conjugated thereto which provide functionality as STING agonist. Such groups, may, for example, be nucleoside analogs and/or nucleotide analogs as described above. Nucleoside analogs used herein may, for example, be a cytidine analog or a derivative of a cytidine analog that is active as a STING agonist. In a number of embodiments, the cytidine analog is gemcitabine, azacitidine, decitabine, zebularine, or cytarabine (also known as cytosine arabinoside or ara-C), or a derivative of such cytidine analogs which is active as a STING agonist (inherently or when incorporated into an amphiphilic polymer hereof). Additionally, one or more C-C chemokine receptor type 2 (CCR2) antagonists is incorporated within the nanostructures. The CCR2 antagonist(s) may, for example, include PF-04136309 (Pfizer; N-(2-((S)-3-(((1r,4S)-4-hydroxy-4-(5-(pyrimidin-2-yl)pyridin-2-yl)cyclohexyl)amino)pyrrolidin-1-yl)-2-oxoethyl)-3-(trifluoromethyl)benzamide), PF-04634817 (Pfizer), BMS 813160 (Bristol-Myers Squibb; N-[(1R,2S,5R)-5-[(1,1-dimethylethyl)amino]-2-[(3S)-3-[[7-(1,1-dimethylethyl)pyrazolo[1,5-a]-1,3,5-triazin-4-yl]amino]-2-oxo-1-pyrrolidinyl]cyclohexyl]-acetamide), BMS-687681 (Bristol-Myers Squibb), BMS-741672 (Bristol-Myers Squibb), Bindarit (Angelini Pharma; 2-methyl-2-[[1-(phenylmethyl)-l-H-indazol-3-yl] methoxy]-propanoic acid), CCX872 (ChemoCentryx), CCX140 (ChemoCentryx), RDC018 (GlaxoSmithKline), DMX-200 (Dimerix; repagermanium or Bis(2-carboxyethylgermanium(IV) sesquioxide)), AZD2423 (Astra Zeneca), Cenicriviroc (Abbvie; (5E)-8-[4-(2-Butoxyethoxy)phenyl]-1-(2-methylpropyl)-N-{4-[(S)-(1-propyl-1H-imidazol-5-yl)methanesulfinyl]phenyl}-1,2,3,4-tetrahydro-1-benzazocine-5-carboxamide), CNTX-6970 (Centrexion Therapeutics), JNJ-17166864 (Johnson & Johnson), MK-0812 (Merck), RAP-103 (Creative Bio-Peptides), RO5234444 (Roche), SSR150106 (Sanofi), WXSH0213 (WuXi), and NOX-E36 (NOXXON Pharma).

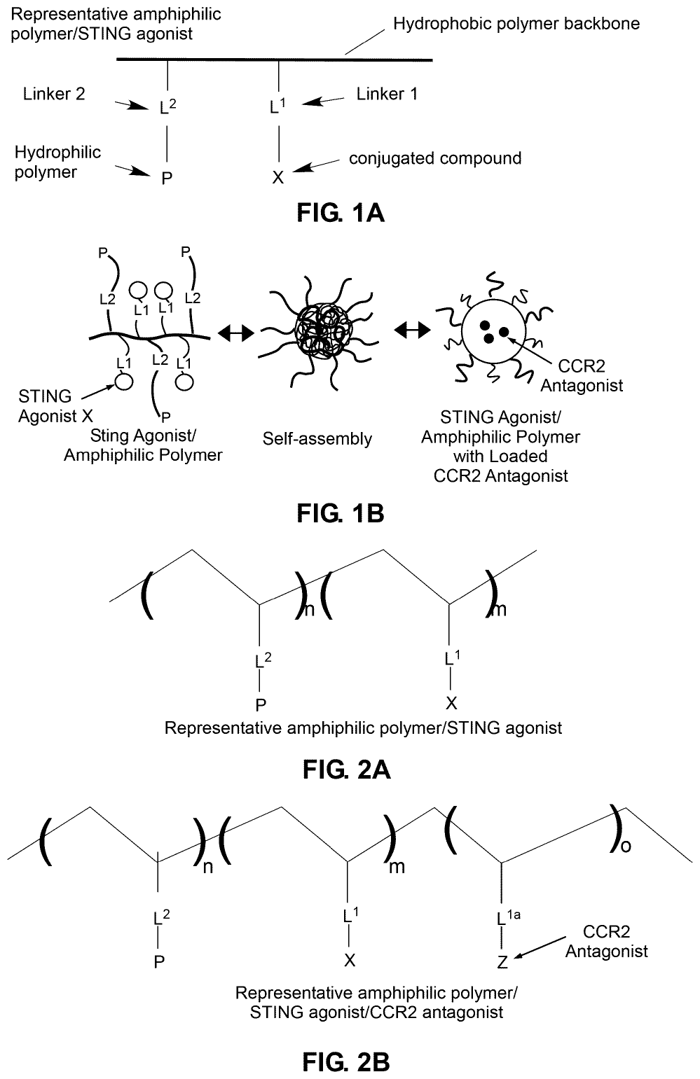

Amphiphilic polymers suitable to self-assemble to form nanostructures such as micelles are, for example, disclosed in PCT International Patent Application Publication Nos. WO 2014/093631, WO 2017/023667, WO 2019/204799, WO 2020/077170, and WO2022/010850, assigned to the assignee of the present invention, the disclosures of which are incorporated herein by reference. In a number of embodiments, the amphiphilic polymers may, for example, include a hydrophobic polymer backbone, and the group(s) which include a conjugated compound (providing STING agonist activity to the polymer/conjugate) may be attached to the hydrophobic polymer backbone as pendant groups. Another plurality of pendant groups may be attached to the hydrophobic polymer backbone which include at least one hydrophilic polymer. Likewise, in other embodiments, a further plurality of pendant groups including a conjugated CCR2 antagonist may be attached to the hydrophobic polymer backbone. FIG. 1A illustrated schematically an embodiment of an amphiphilic polymer hereof. The self-assembly of a plurality of the polymers of FIG. 1A into a nanostructures loaded with a CCR2 antagonist is illustrated schematically in FIG. 1B. FIGS. 2A and 2B illustrate schematically embodiments of amphiphilic polymers hereof wherein the polymer includes a pendant STING agonist X in FIG. 2A and includes a pendant STING agonist X and a pendant CCR2 antagonist Z in FIG. 2B. In general, it is desirable to physically load the CCR2 antagonist into nanostructures formed by amphiphilic polymers hereof. In that regard, chemical bonding or covalently attaching the CCR2 antagonist in certain embodiments may enlarge the nanostructures/micelles formed via self-assembly of the polymers.

In the illustrated embodiments of FIGS. 1A through 2B and other embodiments hereof, the amphiphilic polymer may, for example, be formed via radical polymerization to have a hydrophobic polymer backbone. Synthesis amphiphilic polymers with a hydrophobic backbone and pendant group is, for example, described in PCT International Patent Application Publication No. WO 2020/077170. The hydrophobic polymer backbone may, for example, be formed via a radical polymerization such as a free radical polymerization or via a controlled or living radical polymerization (such as a reversible-deactivation radical polymerization or RDRP). The polymer backbone may, for example, be an alkylene polymer backbone.

Reversible-Deactivation Radical Polymerization (RDRP) procedures include, for example, Nitroxide Mediated Polymerization (NMP), Atom Transfer Radical Polymerization (ATRP), and Reversible Addition Fragmentation Transfer (RAFT) and others (including cobalt mediated transfer) that have evolved over the last two decades. RDRP provide access to polymers and copolymers comprising radically polymerizable/copolymerizable monomers with predefined molecular weights, compositions, architectures and narrow/controlled molecular weight distributions. Because RDRP processes can provide compositionally homogeneous well-defined polymers, with predicted molecular weight, narrow/designed molecular weight distribution, and high degrees of α- and ω-chain end-functionalization, they have been the subject of much study, as reported in several review articles and ACS symposia. See, for example, Qiu, J.; Charleux, B.; Matyjaszewski, K., Prog. Polym. Sci. 2001, 26, 2083; Davis, K. A.; Matyjaszewski, K. Adv. Polym. Si. 2002, 159, 1; Matyjaszewski, K., Ed. Controlled Radical Polymerization; ACS: Washington, D.C., 1998; ACS Symposium Series 685. Matyjaszewski, K., Ed.; Controlled/Living Radical Polymerization. Progress in ATRP, NMP, and RAFT; ACS: Washington, D.C., 2000; ACS Symposium Series 768; and Matyjaszewski, K., Davis, T. P., Eds. Handbook of Radical Polymerization; Wiley: Hoboken, 2002, the disclosures of which are incorporated herein by reference.

In a number of representative polymers hereof, a hydrophobic polymer backbone may be formed via radical polymerization of a variety of radically polymerizable monomers. Such monomers may, for example, include pendant groups as described above prior to polymerization. Alternatively, such pendant groups may be attached after polymerization. Representative monomers for use herein include styrene, acrylic acid, methacrylic acid, acrylonitrile, vinyl monomers and their derivatives. In a number of embodiments, the degree of polymerization for hydrophobic polymers hereof is, for example, less than 500, less than 200 or less than 100.

In the representative embodiments illustrated in FIGS. 1A through 2B, the conjugated compound(s)/group(s) (which when conjugated within the amphiphilic polymer provide functionality/activity as a STING agonist) are illustrated on a pendant chain and are represented by X. The hydrophilic polymer group(s) are represented by P. More than one conjugated compound/group X may be included in a single pendant chain, and more than one hydrophilic polymer P may be included in a single pendant chain. A plurality of such pendant chains may be attached to the hydrophobic polymer backbone. In FIG. 2A, m and n may, for example, independently represent integers as known in the polymer arts. Likewise, in FIG. 28, m, n, and o may, for example, independently represent integers as known in the polymer arts. Referring to FIG. 2A and FIG. 2B, for example, at least one of the first plurality of pendant groups may be attached to the hydrophobic polymer backbone via a linking moiety. The linking moiety may include at least one group interactive via π-π stacking. The pendant group(s) X may, for example, be attached to the hydrophobic polymeric backbone via a first linking group (L¹). The first linking group (L¹) may, for example, include at least a first group which is interactive via π-π stacking. The first linking group (L¹) may be absent and the group X may be directly attached to the hydrophobic polymer backbone. The second plurality of pendant groups may also be attached to the hydrophobic polymer backbone via a second linking group (L²). The second linking group (L²) may be absent and the hydrophilic polymer may be directly attached to the hydrophobic polymer backbone. The second linking group (L²) may, for example, independently include at least a second group interactive via π-π stacking. The first linking group (L¹) and/or the second linking group (L²) may, for example, include an aromatic group. The third plurality of pendant groups may also be attached to the hydrophobic polymer backbone via a third linking group (L^{1a}). The third linking group (L^{1a}) may be absent and the CCR2 antagonist Z may be directly attached to the hydrophobic polymer backbone. The third linking group (L^{1a}) may, for example, independently include at least a second group interactive via π-π stacking. The first linking group (L¹), the second linking group (L²), and/or the third linking group (L^{1a}) may, for example, include an aromatic group. In general, aromatic groups are cyclic molecules including resonance bonds that exhibit increased stability compared to other geometric or connective arrangements with the same set of atoms. Aromatic groups include, for example, benzyl and naphthyl groups. In a number of embodiments hereof, aromatic groups hereof are benzyl groups. In embodiments in which a CCR2 antagonist Z is conjugated to the hydrophobic polymer backbone, a linker group such as linking group (L^{1a}) may, for example, be used.

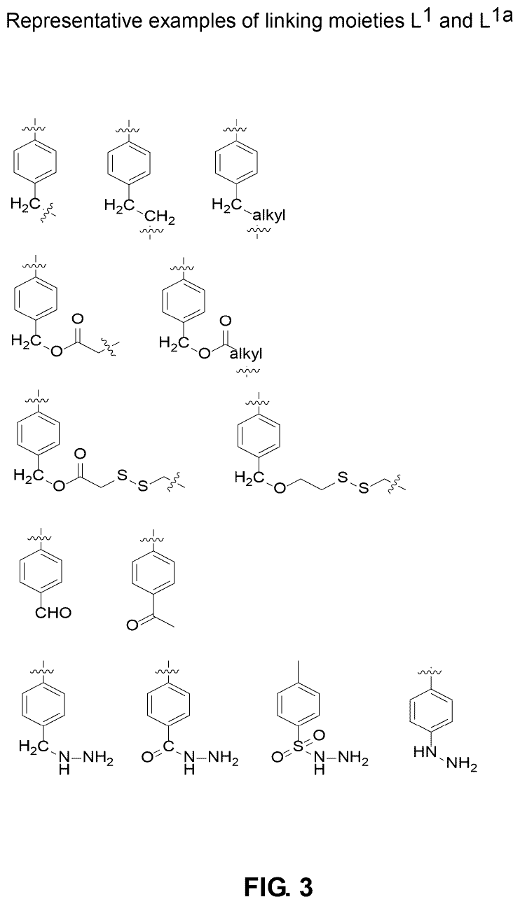

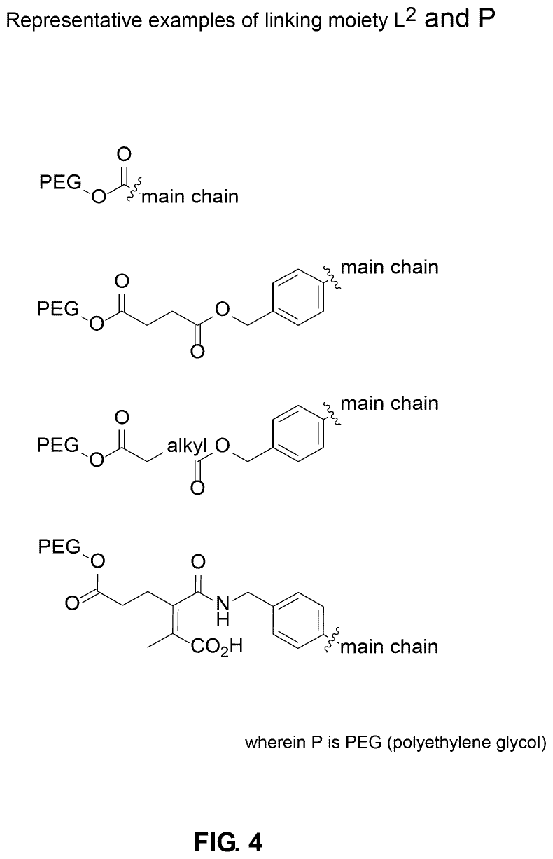

In a number of embodiments, linking moiety L¹ and/or L^{1a} may independently include a group or moiety that is labile in vivo. The group or moiety that is labile (in vivo) may, for example, include at least one of a hydrolytically labile group, a reductive sensitive linkage, a pH-sensitive linkage, a ROS-sensitive linkage, or an enzyme/protease-sensitive linkage. The labile linking group may, for example, be labile under acidic pH conditions. The pH sensitive or acid-labile bond may, for example, include a carboxydimethyl maleate, a hydrazine, an imine, an acetal, an oxime, a silyl ether, a cis-asonityl, a ketal or another pH or acid-labile bond or linkage. A labile bond that is sensitive to acidic conditions may be used to cleave the pendant group in, for example, an acidic tumor environment. In a number of embodiments, the labile linking group is sensitive to reductive such as a disulfide bond. In a number of embodiments, the hydrolytically labile group includes an ester group, an orthoester group, a thioether-ester group, an anhydride group, an amide group (for example, peptide groups), or a carbonate group. ROS-sensitive labile bonds or linkages include, for example, a thioketal bond. An enzyme or protease-sensitive bond or linkage includes, for example, a protease cleavable peptide including the sequence CGLDD which is labile in response to the presence of matrix metalloproteinases MMP-2 or MMP-9. As described above, the linking moiety may, for example, further include at least one group which is interactive via π-π stacking. The at least one group interactive via π-π stacking may, for example, include an aromatic group (for example, a benzyl group). Representative examples of linking moiety L¹, L^{1a}, and L² are illustrated in FIGS. 3 and 4.

The hydrophilic oligomer or the hydrophilic polymer may, for example, be selected from the group consisting of a polyalkylene oxide, a polyvinylalcohol, a polyacrylic acid, a polyacrylamide, a polyoxazoline, a polysaccharide, a polypeptide and biocompatible, water-soluble polymers including sulfoxide functionality. Such hydrophilic polymer and other hydrophilic polymer are known for use in creating hydrophilic domains in amphiphilic compounds which may self-assemble to form nanostructures. In a number of embodiments, the at least one hydrophilic polymer is a polyalkylene oxide. The polyalkylene oxide may, for example, be a polyethylene glycol (PEG). A polyethylene glycol or other hydrophilic polymer hereof may, for example, have a molecular weight of at least 500 Da. In a number of embodiments, the polyethylene glycol of other hydrophilic polymer hereof has a molecular weight in the range of 100 Da to 5 KDa or in the range of 500 Da to 2 KDa. In a number of studies hereof, the polyalkylene oxide PEG was used in forming representative amphiphilic compounds hereof. Other biocompatible hydrophilic compounds, including those described above, are known for medicinal use, including in self-assembling formation of nanostructures, and may be used in place of or in addition to PEG in amphiphilic polymers hereof.

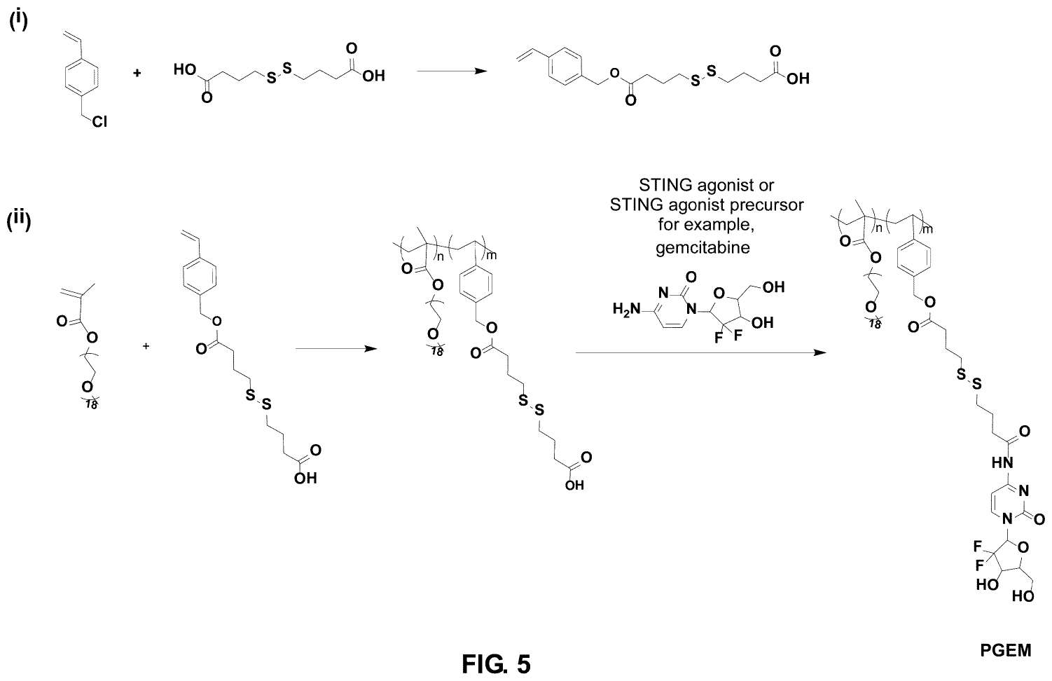

In a representative synthetic scheme illustrated in FIG. 5 of a representative amphiphilic polymer hereof, vinyl benzyl monomers with a labile disulfide linkage (VD monomer) were synthesized via reaction of vinylbenzyl chloride and 4,4′-Dithiodibutyric acid. Then, POEG-co-PVD polymer was synthesized by RAFT co-polymerization of VD monomer and OEG950 monomer. POEG-co-PVDGEM polymer was obtained by conjugating the representative nucleoside analog gemcitabine or GEM to the POEG-co-PVD polymer backbone using, for example, an EDC/HOBt coupling reaction as known in the chemical arts to from gemcitabine-conjugated polymer PGEM. Other STING agonist or STING agonist molecules or precursors such as other nucleoside analogs and nucleotide analogs may be incorporated via a similar reaction using the same reactive functional groups of different functional groups for covalent attachment as known in the chemical arts. The representative reaction scheme of FIG. 5 is further discussed in PCT International Patent Application Publication No. WO 2020/077170. The reaction scheme of FIG. 5 may be readily altered to synthesize polymers including pendant groups with STING agonists other than GEM or to include pendant groups including one or more CCR2 antagonists.

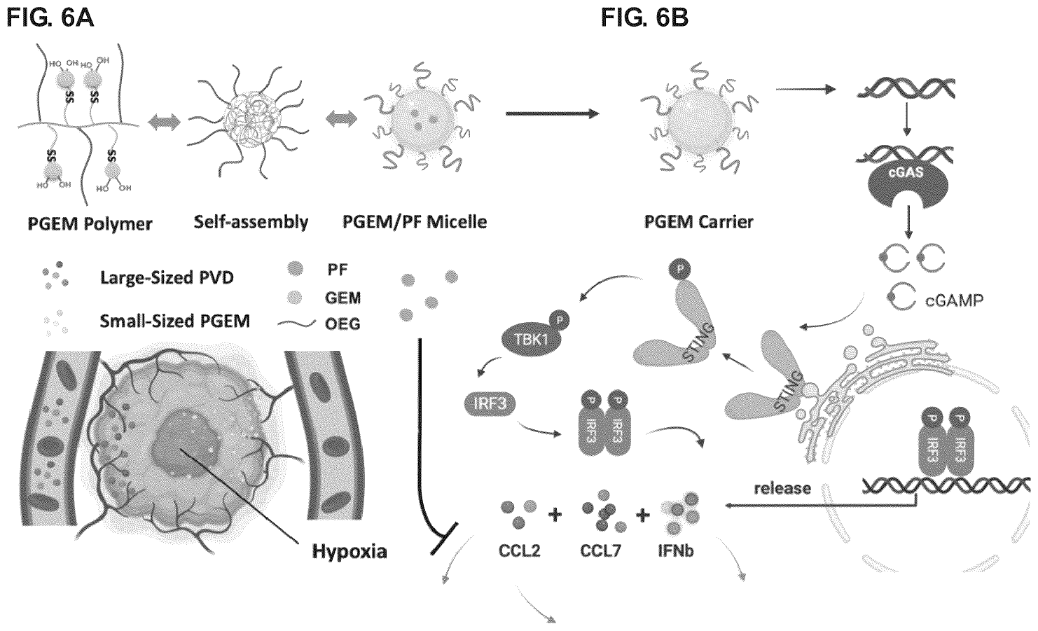

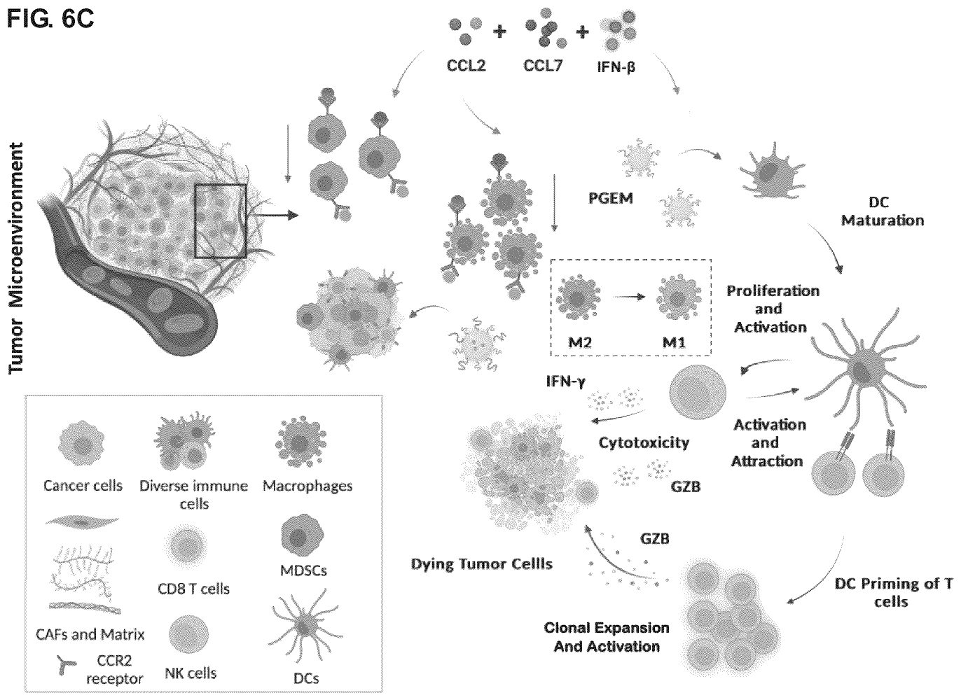

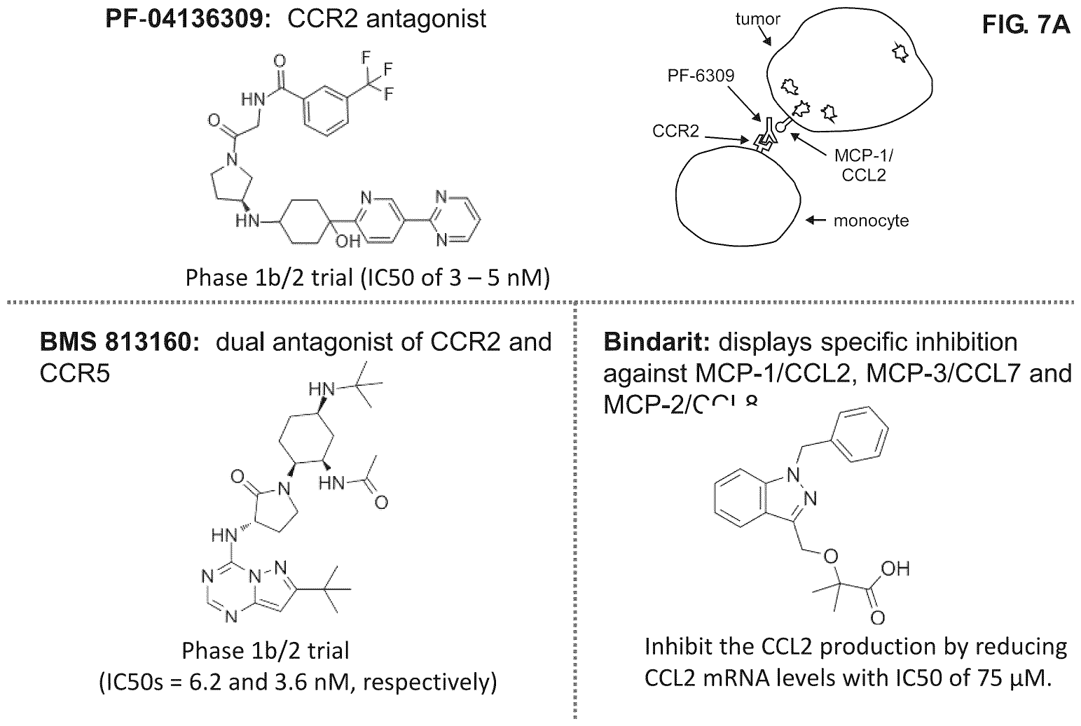

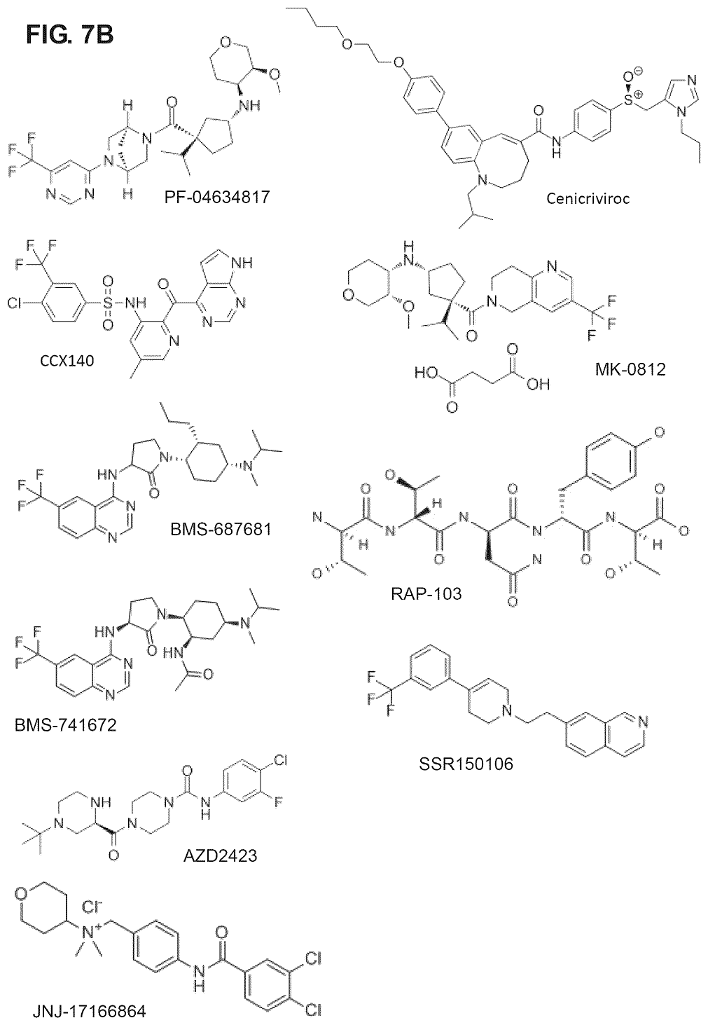

Without limitation to any mechanism, FIGS. 6A through 6C illustrates a proposed scheme or mechanism for the therapeutic effects of a PGEM (or other amphiphilic polymer including a STING agonist) formulation hereof including a CCR2 antagonist (PF-04136309, alternatively PF-6309 or PF) loaded into nanostructures formed by the PGEM polymer. The potential of PGEM/PF in sensitizing PDAC to programmed death-I (PD-1) immune checkpoint blockade was investigated as described further below. In the embodiment of FIGS. 6A through 6C, the CCRS antagonist PF is loaded into PGEM. Other studied CCR2 antagonists include BMS 813160 and Bindarit as illustrated in FIG. 7A. The structures of further CCR2 antagonists for use herein are illustrated in FIG. 7B. In preliminary, non-optimized studies, the best results were obtained with PF.

GEM, which is a cytidine analogue, is the first-line chemotherapeutic drug for the treatment of pancreatic cancer and works through inhibiting DNA replication. GEM was thus chosen as a representative STING agonist for a number of studies hereof. However, limited clinical benefits from GEM have been achieved because of its inefficient delivery and the rapid degradation by cytidine deaminase (CDA). Nanotechnology may offers the advantages of improving drug delivery, protecting GEM from inactivation by deaminase and reducing the toxicity. However, PDAC is known to be poorly vascularized with small pore sizes (˜50-60 nm), which limits the extravasation of large nanoparticles. In addition, as described above, PDAC has dense stroma that further limits the penetration of large nanoparticles into the tumor core. Nab-paclitaxel has been reported to destroy the cancer-associated fibroblasts (CAF) and increase the drug penetration and intratumoral accumulation. However, the stroma depletion could enhance the spread of tumor cells, leading to compromised therapeutic efficacy. Increasing evidence has shown that smaller nanoparticles (530 nm) were much more effective in penetrating stroma-abundant pancreatic tumors without destroying the stroma.

Conjugation of GEM to PVD backbone dramatically decreases the particle size from ˜160 nm to ˜13 nm, yielding an ultra-small prodrug polymeric carrier (PGEM). The structures of the PVD and PGEM polymers were characterized by FT-IR. Compared to PVD, PGEM showed strong broad O—H stretching at 3600-3100, indicating the successful conjugation of GEM to PVD backbone.

The tumor penetration of PGEM nanocarrier was first evaluated by using Panc02 cell- and KPC cell-derived tumor spheroids. To mimic the tumor microenvironment in vivo, the spheroids were composed of both the tumor cells and collagen matrix. The spheroids were treated with rhodamine-loaded PGEM and PVD micelles, respectively, for visualization by Confocal Z-stacks scanning with 20 μm Z-intervals (20-80 μm). PVD/Rhodamine micelles (˜160 nm) were mainly localized around the peripheral area with essentially no signals in the core. In contrast, the PGEM micelles (˜13 nm) showed significantly stronger ability to penetrate the tumor and the signals were distributed throughout the entire spheroids.

PGEM has been shown to significantly inhibit tumor growth in several tumor models, including Panc02, CT26 and PDX models. To gain mechanistic insights for the PGEM-mediated anti-tumor response, RNA sequencing (RNA-Seq) was performed on Panc02 tumor tissues treated with saline, GEM, and PGEM, respectively. Four hundreds and eighty-nine (489) genes were significantly downregulated and 416 genes were significantly upregulated after GEM treatment, while 407 genes were significantly downregulated and 461 genes were significantly upregulated after PGEM treatment (Cuffdiff P<0.05). Through Gene Set Enrichment Analysis (GSEA), it was found that PGEM treatment significantly downregulated several signaling pathways critically involved in the initiation and progression of PC, including MTORC1_SIGNALING, HYPOXIA, HEDGEHOG SIGNALING, NOTCH_SIGNALING and KRAS_SIGNALING.

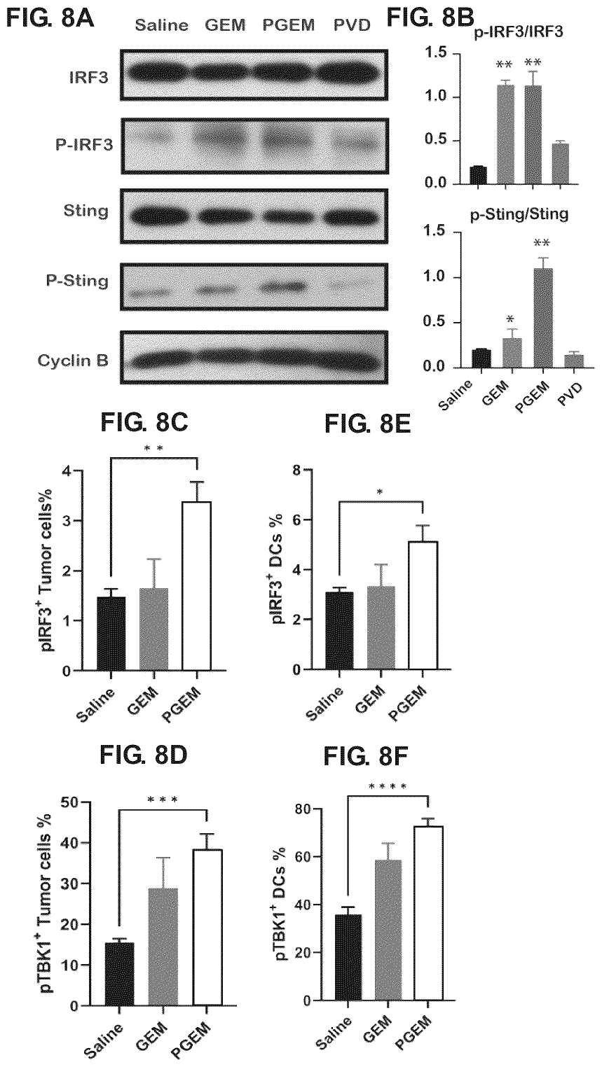

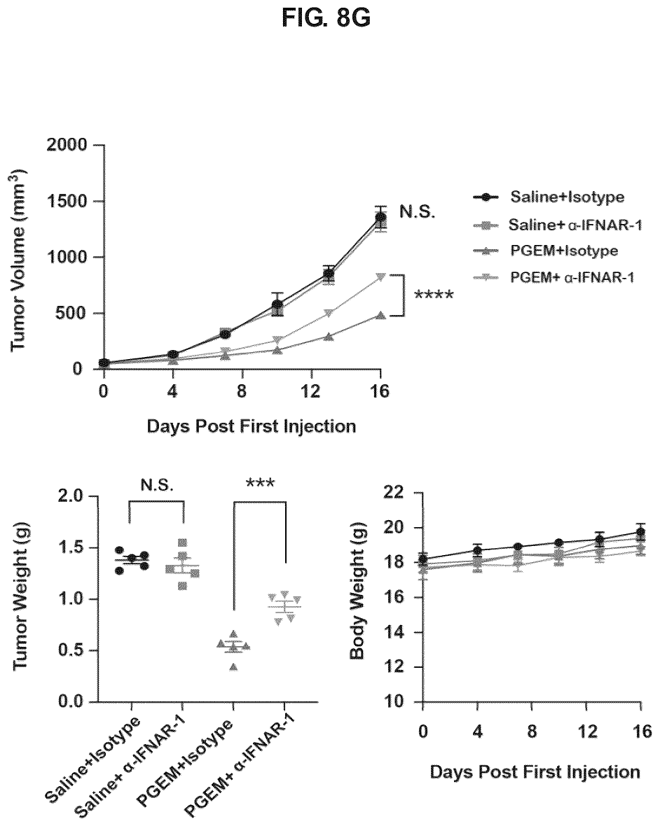

Cytosolic DNA sensing signaling, interferon-alpha response, and NFKB signaling pathway were upregulated by PGEM comparing to GEM treatment. As the binding of cytosolic DNA with cGAS has been shown to activate cGAS-STING pathway and trigger downstream pathways such as type I interferon and nuclear factor κB (NF-κB) signalings, it was hypothesized that PGEM might activate cGAS-STING pathway. To test this hypothesis, the presence of cytosolic DNA and its colocalization was evaluated with cGAS by picogreen staining and immunofluorescence. Treatment with GEM or PGEM significantly increased the signals of cGAS-bound micronuclei in Panc02 cells compared with control and PVD-treated groups. The expression of STING pathway-related proteins was then evaluated after various treatments by Western blot. As shown in FIG. 8A, GEM or PGEM treatment significantly induced IRF3 and STING phosphorylation in Panc02 cells. The normalized protein levels of phosphorylated IRF3 (pIRF3) and phosphorylated STING (pSTING) were significantly higher in PGEM- and GEM-treated groups compared with saline and PVD polymer-treated groups (FIG. 8B). Those results indicated that treatment with GEM or PGEM was able to activate STING pathway in Panc02 tumor cells. Accumulating evidence has shown that cGAS-STING pathway can be triggered by DNA damage. Therefore, whether PGEM activated cGAS-STING pathway through inducing DNA damage was evaluated by using γH2AX foci as a DNA double strand break (DSB) marker. Formation of γH2AX foci was induced in both GEM- and PGEM-treated Panc02 cells. However, saline- and PVD polymer-treated Panc02 cells displayed no γH2AX foci. These findings suggested that PGEM and GEM potently activated the double-strand DNA damage in Panc02 tumor cells in vitro, resulting in subsequent activation of cGAS-STING pathway signaling.

It has previously been reported that the cGAS-STING pathway could be activated both in tumor cells and dendritic cells (DC), and activation of STING-IRF3 pathway in DCs is required for antitumor CD8⁺ T cell response. Thus, whether PGEM activated the STING pathway was investigated in both types of cells by flow cytometric analysis of tumor tissues treated with saline, GEM, and PGEM in GFP-Panc02 syngeneic tumor model. As shown in FIG. 8C, PGEM treatments significantly increased the proportion of phosphorylated IRF3 (Ser396, pIRF3⁺)- and phosphorylated TBK1-positive (Ser172, pTBK1⁺) tumor cells, suggesting the activation of STING-TBK1-IRF3 signaling in tumor cells in vivo, which was consistent with in vitro results of studies hereof. In addition, PGEM treatment significantly increased the proportion of pTBK1- and pIRF3-positive CD11c⁺ CD11b⁻ DCs (FIG. 8D).

To investigate if the cGAS-STING activation by PGEM led to the production of type I IFN, the in vitro and in vivo expression of IFNβ, a downstream molecule in the STING signaling pathway, was assessed. PGEM significantly increased the mRNA levels of IFNβ in both cultured Panc02 cells and harvested tumor tissues. Whether the efficacy of PGEM therapy is dependent on type I IFNs was also evaluated. The type I IFNs signaling was blocked using antibody against the type I interferon receptor (IFNAR-1). As shown in FIG. 8E, PGEM treatment in the presence of blocking antibody led to substantial reduction in therapeutic efficacy of PGEM, demonstrating that PGEM therapy is dependent on type I IFNs. Taken together, and without limitation to any mechanism, these results indicated that PGEM treatment activated the cGAS-STING pathway and induced type I IFN production, which contributed to the antitumor activity of PGEM. It is believed that this is the first study to report that a GEM-based polymer can activate the cGAS-STING pathway. It has been reported that another polymer bearing a seven-membered ring with a tertiary amine (PC7A) activated STING pathway through the polymer-induced formation of STING-PC7A condensates, which was cGAS independent. Without limitation to any mechanism, it appears in the present case that the STING activation is associated with DNA damage, which facilitates the cytosolic retention of cGAS.

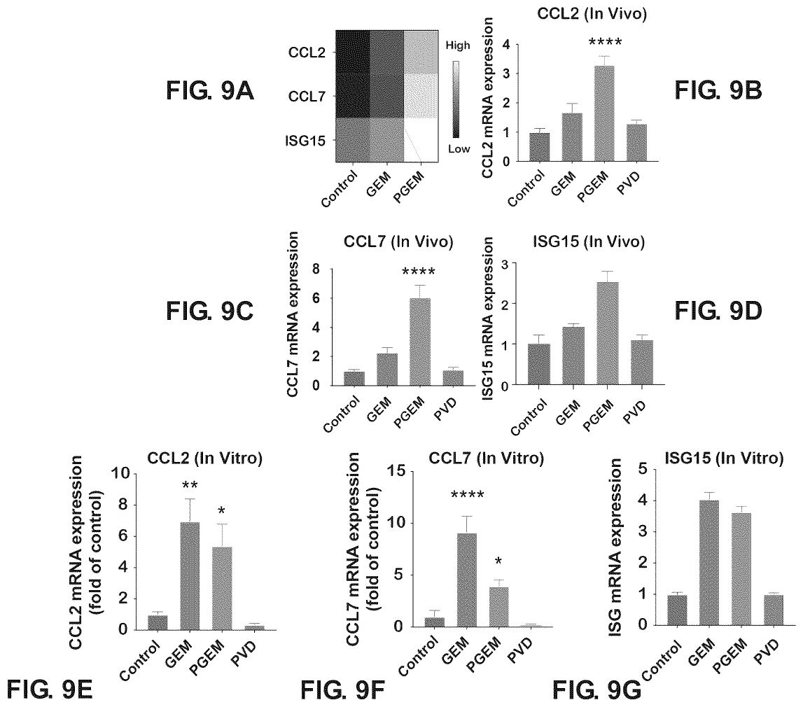

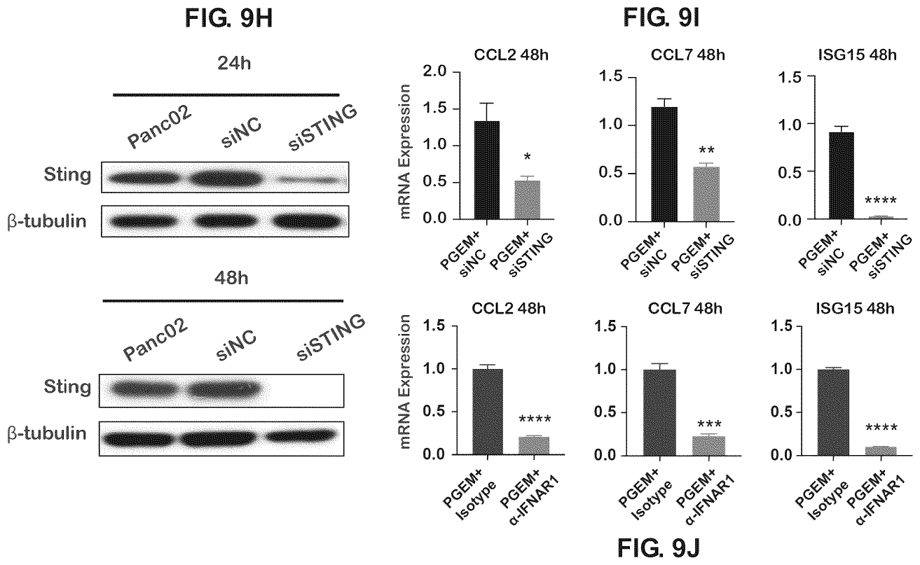

The RNAseq data clearly showed that PGEM downregulated gene sets involved in PC pathogenesis and activated cGAS-STING innate immune pathway. However, upregulation of several genes that have been shown to both enhance tumor growth and promote an immunosuppressive TME including CCL2 and CCL7 was also observed after treatment with either GEM or PGEM (FIG. 9A). GEM and PGEM treatments also upregulated the expression of interferon-stimulated gene 15 (ISG15), which is highly correlated with GEM resistance. To validate the RNAseq data, the expression of CCL2, CCL7 and ISG15 was evaluated by qRT-PCR in vivo and in vitro (FIGS. 9B through 9G). PGEM treatment was more effective in upregulating CCL2, CCL7 and ISG15 expression compared with free GEM in the Panc02 tumor tissues (FIGS. 9B through 9D), while treatment of PVD polymer backbone without GEM showed no obvious changes in these chemokines, indicating that the upregulation is GEM-specific. Notably, PGEM and free GEM similarly induced the expression of CCL2, CCL7 and ISG15 (FIGS. 9E through 9G) in cultured Panc02 cells with free GEM being more effective. Without limitation to any mechanism, this is likely due to the slow release of GEM from PGEM in cultured cells during the limited incubation time while PGEM improves the bioavailability of GEM in tumors in vivo by protecting GEM from enzymatic degradation.

It has been reported that radiation caused CCL2 and CCL7 induction through STING activation. In addition, ISG15 could also be induced by type I interferon (IFN). It was hypothesized that activation of cGAS-STING pathway is involved in the PGEM-mediated chemokine induction. To test this hypothesis, whether knockdown of STING by STING siRNA (siSTING) would decrease the PGEM-induced expression of CCL2 and CCL7 was investigated. The efficiency of STING protein expression was confirmed by Western blot in Panc02 cells following treatment with siSTING for 48 h (FIG. 9H). As shown in FIG. 9I, treatment with siSTING significantly attenuated the PGEM-induced expression levels of CCL2, CCL7 and ISG15. It was also evaluated whether the induction of CCL2, CCL7 and ISG15 by PGEM was related to the type I IFNs. As shown in FIG. 9J, the expression levels of CCL2, CCL7 and ISG15 in the PGEM-treated Panc02 cells were significantly downregulated in the presence of IFNAR1 neutralization antibody compared to isotype control. These data indicated that the PGEM induced CCL2, CCL7 and ISG15 were STING- and type I IFNs-dependent.

The above data are consistent with the notion that the cGAS-STING pathway is a double-edged sword and the outcome is context-dependent. On the one hand, cGAS-STING pathway has the potential to elicit innate and adaptive immune responses, both of which are critical for cancer immunotherapy. The activation of STING drives the production of cytokines Type I IFNs, which promote the generation of cytotoxic T cell responses as well as type I T helper cell (Th1)-biased responses. Furthermore, type I IFNs promote the activation and functional maturation of DCs, thereby facilitating antigen presentation to CD4′ T cells as well as antigen cross-presentation to CD8⁺ T cells. On the other hand, STING activation has been found to promote the proliferation of metastatic cells and chemoresistance in several type of cancers such as breast cancer and lung cancer. In addition, evidence has shown that STING activation induced CCL2 and CCL7 expression, which was associated with immunosuppressive TME due to M-MDSC infiltration, leading to tumor radio-resistance. STING agonist cyclic diadenyl monophosphate (CDA) was also reported to cause induction of other immune regulatory pathways such as cyclooxygenase-2 (COX2) and PD-1 in the TME. Blocking this feedback mechanism via COX-2 inhibition synergized with CDA in inhibiting tumor growth in Lewis lung carcinoma. Although the negative feedback mechanisms of STING activation and their biological consequences are not fully understood, the studies hereof demonstrate that PGEM may function as a polymeric STING agonist. Although STING activation likely contributed to the PGEM antitumor activity through the induction of the type I IFN production, it also triggered the undesired induction of CCL2/CCL7. Thus, the potential of combining PGEM with blockade of CCR2, the receptor shared by both CCL2 and CCL7, was examined.

There are several CCR2 inhibitors/antagonists currently approved by FDA or in clinical trials, such as PF-04136309 (PF), BMS 813160 (BMS) and Bindarit (Bind), but the therapeutic efficacies of all such compounds are limited by their poor solubility and low bioavailability. The PGEM carrier hereof was able to load these inhibitors to form micelles with ultra-small sizes and close-to-neutral zeta potentials, which well-addressed the solubility and systemic delivery issues.

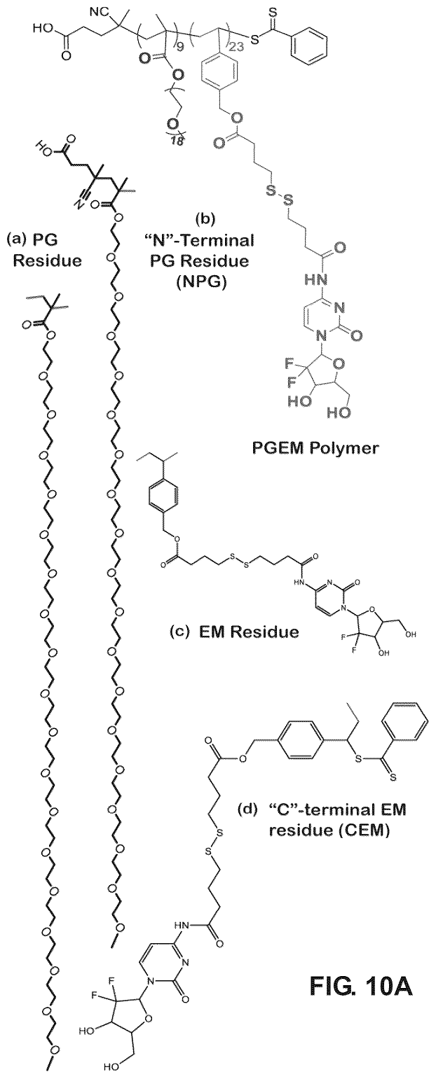

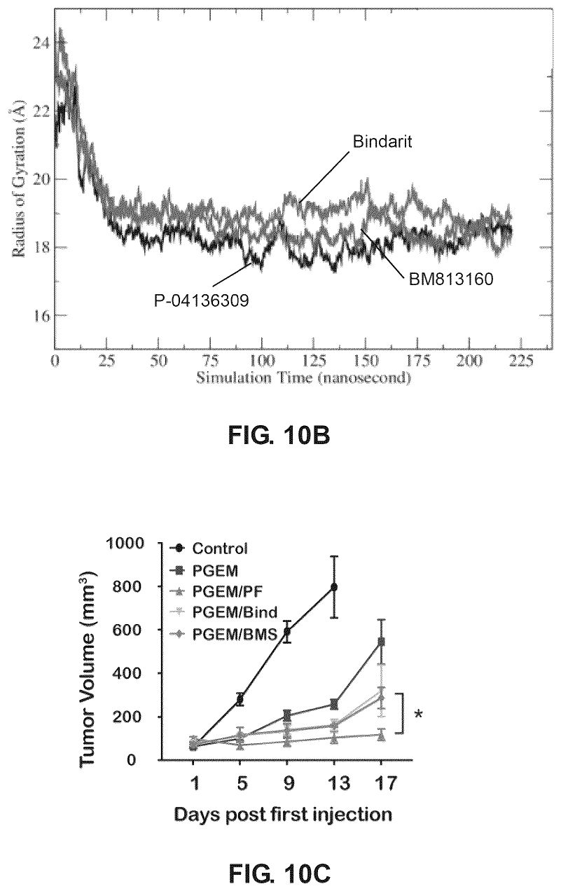

Current drug formulations are largely developed through trial and error with little insight into the biophysical interaction of drug carriers and loaded drugs. For a rational selection of a CCR2 antagonist that is “optimally” paired with the PGEM-based drug carrier, a state-of-the-art molecular dynamic simulations was applied to study how these three inhibitors interacted with the PGEM polymer. Firstly, a series of sequential molecular dynamics (MD) simulations was used to generate the micelle-like structure formed by a single copy of PGEM polymer, which consists of 32 residues from 4 residue types, including PG (FIG. 10A(a)), NPG (N-terminal PG) (FIG. 10A(b)), EM (FIG. 10A(c)), and CEM (C-terminal EM) (FIG. 10A(d)). To accurately describe the properties of biomolecules in aqueous environment, solvent effects were also considered in the MD simulation. Therefore, the interaction between different CCR2 inhibitors with the PGEM polymer was studied by the combination of Generalized Born molecular dynamics (GBMD) simulation and explicit water MD simulations. Due to highly dynamic attribute of the systems, radii of gyration (RoG) of the polymer and polymer-drug systems were calculated instead of root-mean-square deviation (RMSD), to monitor the conformational change and polymer-drug complex formation (FIG. 10B). For each MD trajectory, the snapshot which had the smallest RMSD to the average structure was selected as the representative conformation. The representative conformations of the polymer itself and polymer-drug complexes were determined. PF-04136309 can interact with the inside hydrophobic domain of the micelle-like structure by forming T-shape and parallel displaced π-π interactions, while the other two drugs have weak interaction with the hydrophobic domain of the polymer.

The “three-trajectories” protocol was used to calculate the binding free energy between the polymer and drug molecules. The MM-PBSA-WSAS free energies of the polymer, the drugs and polymer-drug complexes and the binding free energies were determined. The binding free energies between 8 copies of the drug and the polymer are 5.14, −18.21 and −38.10 kcal/mol for Bindarit, BMS813160, and PF-04136309, respectively. One can also obtain the binding free energies between the polymer and one copy of drug molecule, which are 0.64, −2.28 and −4.76 kcal/mol, correspondingly. The result of MM-PBSA-WSAS free energy analysis is consistent with the MD structures, further confirming that PF-04136309 has much stronger interaction with the PGEM polymer.

It was also found that PGEM/PF demonstrated better stability profile in the presence of serum compared to other formulations. The PGEM/PF micelles remained stable without significant changes in sizes in 50% FBS for 48 h. PGEM/BMS micelles and PGEM/Bind micelles were somewhat less stable and showed increased size at 24 h.

It was further assessed the anti-tumor activity of PGEM loaded with PF, BMS, and Bind in the Panc02 tumor bearing mice, respectively. All the formulations showed no obvious toxicity as evident from the slight increases in the body weights of the treated mice. All three combinations were effective. However, among the three agents tested, PF-04136309 showed the best combination effect with PGEM (FIG. 10C). The in vitro cytotoxicity of PGEM/PF was also compared with those of other two formulations (PGEM/BMS and PGEM/Bind). No significant difference was observed in the cytotoxicity among the three formulations, further indicating that the higher anti-tumor activity of PGEM/PF was likely attributed to the better formulation stability in vivo due to the stronger interaction of PF with PGEM. Thus, PF-04136309 was chosen for subsequent studies. The experimental data matched well with the result of MM-PBSA-WSAS analysis. The above study provides a pilot work of proof-of-principle for selection/optimization of drug-drug formulation pairing for P-STING-agonist/CCR2 antagonist-based combination therapies as well as other combination therapies.

The PF release from PGEM/PF formulation was evaluated in vitro by a dialysis method. Free PF was rapidly diffused across the dialysis bag with over 95% being diffused out of dialysis bag within 24 h. In contrast, PGEM/PF micelles showed a more favorable release kinetics of PF, and only 20% was slowly released within 72 h. Due to the chemical conjugation of GEM to the polymer, no detectable GEM was released within 24 h. Whether PGEM nanocarrier could selectively deliver PF into pancreatic tumors and the retention time of PF in the tumor tissues was also quantitatively evaluated via UPLC-MS. Higher amount of PF was detected in the tumors compared to other organs, suggesting that PGEM nanocarrier was able to selectively deliver PF to the tumor sites. Compared to the kinetics of DiR in tumors, PF showed a relatively faster clearance in tumors which may be due to a relatively rapid metabolism of PF in tumor tissues.

Subsequently, the therapeutic efficacy of PGEM/PF was evaluated both in vitro and in vivo. The combination effect of free PF and GEM as well as PGEM/PF formulation in cytotoxicity was first examined in Panc02 cells. Free PF showed slight cytotoxicity in vitro. Compared to free GEM, combination of PF with GEM increased the overall cytotoxicity, indicating that PF could sensitize the pancreatic tumor cells to GEM treatment. Compared to PGEM, incorporation of PF into PGEM carrier significantly enhanced the cytotoxicity at high concentrations.

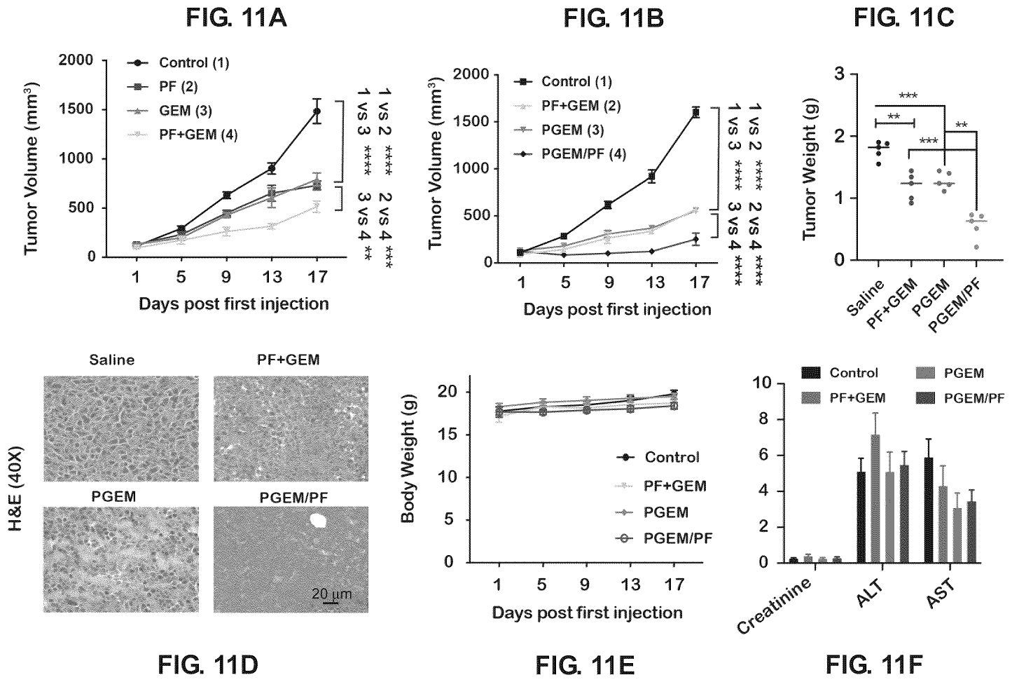

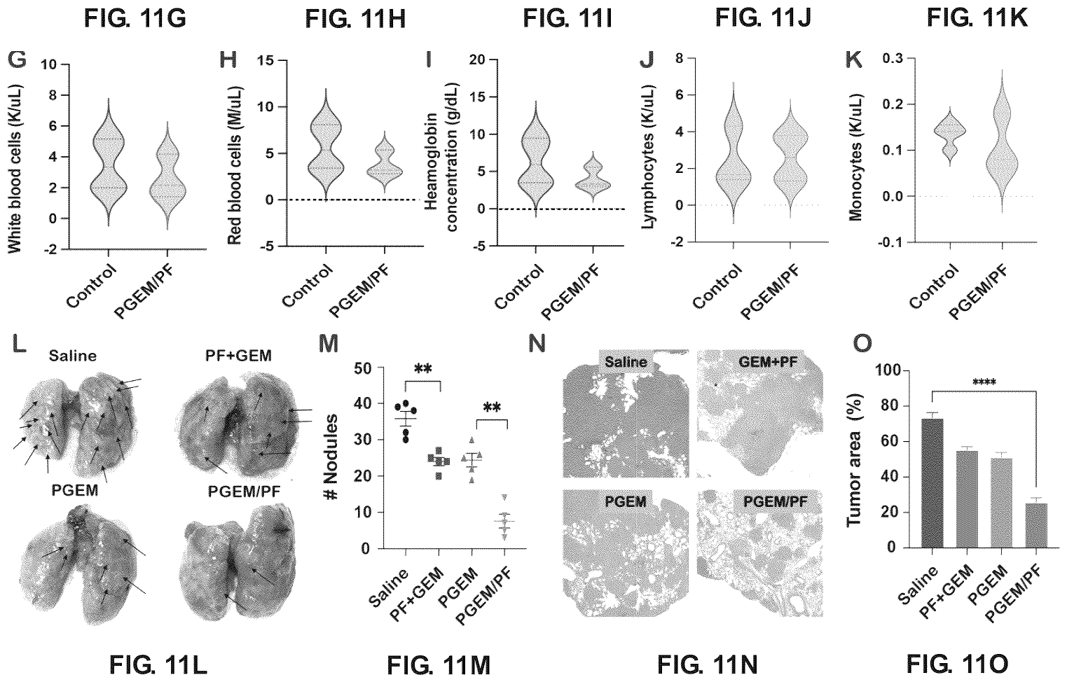

The combination effect of free PF and GEM in vivo was also evaluated. As shown in FIG. 11A, free PF or GEM alone showed modest effect in inhibiting the growth of Panc02 tumor. Combination of the two drugs led to improvement in the overall therapeutic efficacy, indicating their potential combination/synergistic effect. Subsequently, it was evaluated whether loading PF into PGEM carrier further improved the therapeutic efficacy. As shown in the FIG. 11B, PGEM showed a therapeutic efficacy that was comparable to that of free PF and GEM combination. PF formulated in the PGEM carrier significantly enhanced the therapeutic efficacy of PGEM. This result was further confirmed by the lowest tumor weights at the end point (FIG. 11C). The tumor histology was examined at the end of the experiment. As shown in FIG. 11D, H&E-stained tumor sections in the saline-treated group showed typical high density of tumor cells with large nuclei. In comparison, the tumor sections in other treatments showed areas of necrosis. In addition, altered morphology of cancer cells with shrunk nuclei and cell damage was observed. Among all the four treatment groups, PGEM/PF micelles exhibited the most significant intra-tumor tissue damage.

There were no obvious changes in the mouse body weights following various treatments (FIG. 11E). Furthermore, the treatments had no effect on liver and kidney functions as evident from little changes in serum levels of alanine aminotransferase (ALT), aspartate aminotransferase (AST) and creatinine (FIG. 11F). In addition, there were no obvious changes in white blood cell (WBCs), red blood cells (RBCs), hemoglobin concentrations, lymphocytes, monocytes and other immune cells (leukocytes) in peripheral blood after treatment with PGEM/PF in comparison to control group (FIGS. 11G-11K). Finally, no obvious changes were found in histology for all major organs examined including heart, liver, spleen, lung, and kidney after different treatments. The data thus indicated an excellent safety profile of the PGEM/PF at a dosage that demonstrated significant therapeutic efficacy.

As metastasis is one of the leading causes of death in pancreatic cancer patients, the anti-metastasis effect of PGEM/PF was also evaluated in a murine pulmonary metastasis model of pancreatic cancer. As shown in FIG. 11L, PGEM showed an anti-metastasis efficacy that was comparable to that of free PF and GEM combination. PGEM/PF micelles substantially controlled the metastasis of pancreatic cancer. This result was further confirmed by the fewest tumor nodules at the end time point (FIG. 11M). The lung histology was examined at the end of the experiment. As shown in FIGS. 11N and 11O, the PGEM/PF-treated group showed significantly less density of tumor cell infiltration in the lung compared to other groups, further indicating better anti-metastasis effect of PGEM/PF combination.

The enhanced inhibition of tumor growth by PGEM/PF was translated into a prolongation in survival time. Among different treatments, PGEM/PF led to longer survival rate of the mice, further suggesting its significant therapeutic outcomes.

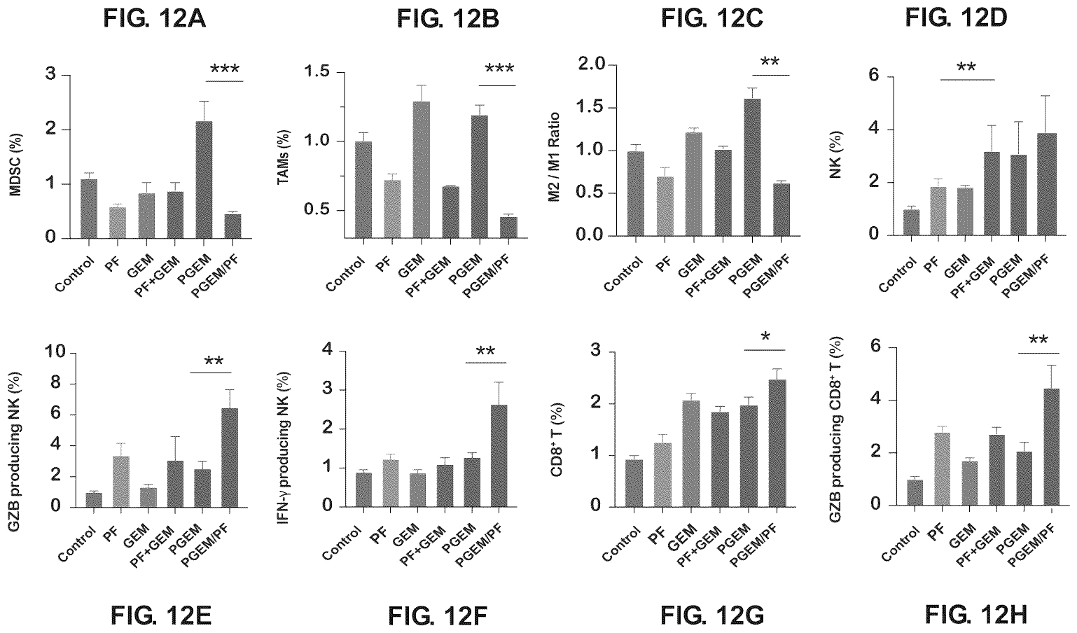

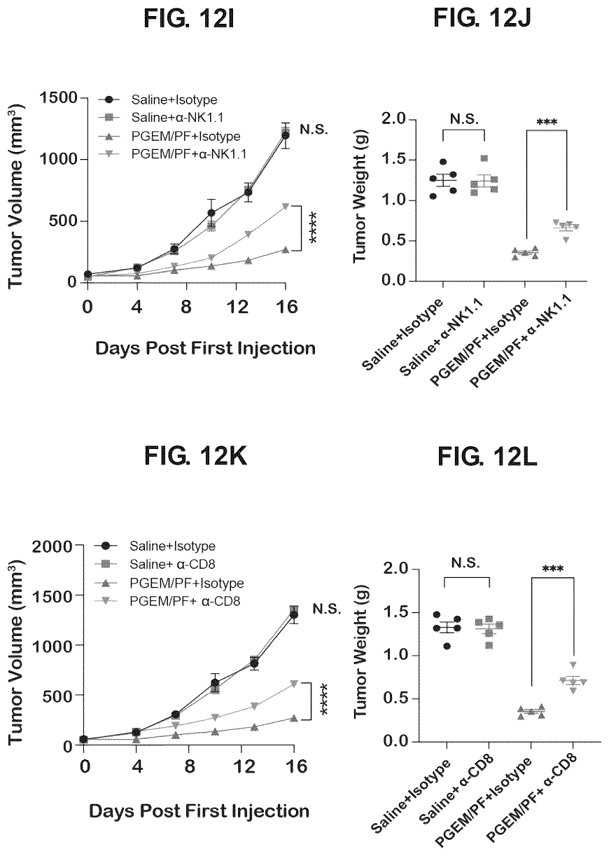

The impact of PGEM/PF on tumor immune microenvironment was further investigated by flow cytometric analysis of harvested tumors after various treatments. As shown in FIGS. 12A-12C, PGEM treatment led to a drastic increase in the percentage of MDSCs and TAMs, which might be attributed to the upregulation of CCL2 and CCL7 as described above. It is also apparent that free PF and PGEM/PF treatments led to a decrease in the percentage of MDSCs and TAMs. Interestingly, PGEM/PF treatment led to a decrease in the percentage of M2 type macrophages, along with an increase in the percentage of M I type macrophages and M1/M2 ratios, suggesting that the tumor infiltrating macrophages were polarized from a tumor-promoting to a tumor-suppressing phenotype. These results indicated that incorporation of PF into PGEM could reverse the MDSCs- and TAMs-immunosuppression induced by the PGEM.

Natural Killer (NK) cells are lymphocytes involved in the early innate immune response. As shown in FIGS. 12D through 12F, there was a significant increase in the total percentage of NK cells in the tumors after the treatments with free PF+GEM combination, PGEM, and PGEM/PF. Moreover, the numbers of IFN-γ⁺ NK and GZB⁺ NK cells were significantly increased in the tumors treated with PGEM/PF in comparison to all the other treated groups. It is known that IFN-γ and GZB are pleiotropic cytokines that can eliminate tumor cells directly and indirectly. Therefore, PGEM/PF treatment increased the number of functional NK cells in the tumors. CD8⁺ T cells are critically involved in the adaptive immune response against cancer. As shown in FIGS. 12G through 12H, all treatments increased the percentages of CD8⁺ T cells in the tumors. Compared to PGEM treatment, incorporation of PF into PGEM carrier further increased the percentages of CD8⁺ T cells in the tumors. In addition, PGEM/PF was highly effective in increasing the number of functional CD8⁺ T cells (GZB CD8⁺ T cells) (FIG. 12H). The above data demonstrated remarkable changes in the immune landscape of PDAC tumors after PGEM/PF treatment.

To characterize the role of NK and CD8⁺ T cells in the significant tumor inhibition effect of PGEM/PF treatment, neutralizing monoclonal antibodies (mAbs) were used to deplete NK and CD8⁺ T cells. Similar to the data shown in FIG. 11B, PGEM/PF treatment led to significant growth inhibition of Panc02 tumors that received IgG isotype controls. In contrast, injection of either anti-NK or anti-CD8 mAb significantly attenuated the tumor growth inhibition in the PGEM/PF-treated mice (FIG. 12I through 12L). This implies that both NK cells and cytotoxic CD8⁺ T cells contributed significantly to the antitumor immunity in PGEM/PF treated mice.

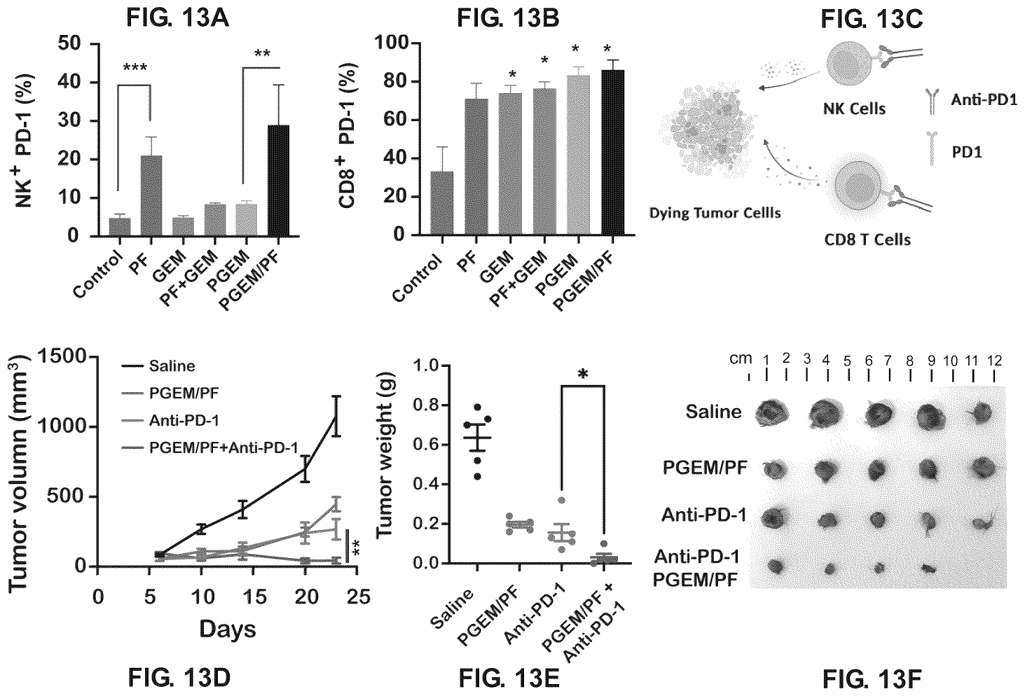

Although PGEM/PF treatment led to an immune-active TME, it also triggered concurrent induction of immune checkpoint molecules. Fluorescence-Activated Cell Sorting (FACS) showed significant up-regulation of PD-1 expression in NK cell and CD8⁺ T cells (as quantified in FIGS. 13A and 13B) in tumors after PGEM/PF treatment. Therefore, it was hypothesized that the tumor growth inhibition after PGEM/PF treatment can be further improved through combination with anti-PD-1 immune checkpoint blockade (ICB) (FIG. 13C). As shown in FIGS. 13D through 13F, compared to PGEM/PF and anti-PD-1 alone, the combination treatment led to significantly improved inhibition effect on tumor growth. One out of the 5 tumors completely regressed, clearly demonstrating the significant therapeutic benefit of combining the two treatments (FIG. 13F).

In summary, in representative studies hereof, the formulations hereof were further found to sensitize solid tumors to programmed cell death protein 1 (PD-1) therapy. Although P(STING agonist)/CCR2 antagonist (for example, PGEM/PF) treatment led to an immune-active tumor microenvironment or TME, it also triggered concurrent induction of immune checkpoint molecules. FACS showed significant up-regulation of PD-1 expression in NK cell and CD8⁺ T cells in tumors after PGEM/PF treatment. It was hypothesized that the tumor growth inhibition after PGEM/PF treatment could be further improved through combination with anti-PD-1 immune checkpoint blockade (ICB). In representative studies, it was determined that, compared to PGEM/PF and anti-PD-1 alone, a combination treatment led to a significantly improved inhibition effect on tumor growth. One out of the 5 tumors completely regressed, clearly demonstrating the remarkable therapeutic benefit of combining the two treatments.

Guided by RNAseq and computer modeling, a new immunochemotherapy formulation (for example, for solid tumors including PDAC) was developed in which a STING agonist and a CCR2 antagonist are co-delivered. The combination formulation may further be combined with an anti-PD-1 therapy. The STING agonist and a CCR2 antagonist may, for example, be co-deliverable via a nanostructure or nanoparticle. In a number of representative embodiments, an immunochemotherapy formulation hereof is formed by incorporating a CCR2 antagonist (for example, PF) into a nanostructure formed by an amphiphilic polymer/STING-agonist drug/prodrug. Such amphiphilic polymers hereof self-assemble to from nanostructures such as micelles.

in a number of representative studies, a representative polymer-STING agonist (polymer-GEM or PGEM) was found to form ultrasmall nanostructures/nanocarriers. The PGEM nanocarrier showed excellent tumor penetration in pancreatic tumor spheroids and in a KPC orthotopic tumor model. The KPC mouse is an established and clinically relevant model of pancreatic ductal adenocarcinoma (PDA). The PGEM carrier itself could activate the cGAS-STING signaling pathway in both pancreatic tumor cells and dendritic cells (DCs), and mediated potent anti-tumor immune response. PGEM was found to promote the recruitment of more tumor-infiltrating NK cells and CD8 T cells and led to increased production of IFN-γ and GZB in those two cell populations. It was also found that PGEM treatment upregulated the expression of chemokines CCL2 and CCL7, which induced immune resistance by recruiting more M2 associated macrophage and MDSCs. Incorporating a CCR2 antagonist such as PF-6309 or other CCR2 antagonists into PGEM carrier alters the tumor immune microenvironment by reversing the immunosuppression mediated by M2 and myeloid-derived suppressor cells (MDSCs) while maintaining the effective tumor-infiltration of NK cells and CD8 T cells, which led to amplified anti-tumor immunity and enhanced anti-tumor response. Furthermore, the representative PGEM/PF formulation sensitized PDAC tumors to anti-PD-1 therapy, and the combination treatment led to significant suppression/eradication of the tumors.

Data from representative studies hereof, using a mouse model of pancreatic cancer, demonstrated that representative nanostructures, nanoparticles or nanocarriers formed, for example, from amphiphilic polymer/STING agonists (for example, a nucleoside-conjugated polymer such as PGEM), formulated as an ultrasmall micellular nanomedicine). The nanostructure/nanomedicine may carry or incorporate a CCR2 antagonist (for example, PGEM+a small molecule inhibitor for CCR2 or CCR2i). The combination nanomedicines hereof were shown to arrest tumor growth more effectively than free STING agonist and the CCR2 antagonist co-administered at equipotent doses. An important component to the enhanced response was intra-tumoral data that showed a shift toward an inflammatory tumor microenvironment following treatment with representative PGEM+CCR2i nanomedicine therapy. Specifically, treatment with representative PGEM+CCR2i nanomedicine reduced the percentage of immunosuppressive innate cell populations, myeloid-derived suppressor cells and type-2 tumor associated macrophages, compared to free gemcitabine and CCR2i co-administration. Notably, populations of cytotoxic natural killer cells producing granzyme B and IFNg, as well as CD8+ T cells and granzyme B producing-CD8+ T cells significantly increased in the tumors of mice treated with PGEM+CCR2i nanomedicine. The enhanced intra-tumoral inflammatory response elicited by PGEM+CCR2i nanomedicine therapy induced upregulation of the membrane protein, programmed-death receptor-i (PD-1), which acts as a co-inhibitory factor of the immune response. When PGEM+CCR2i nanomedicine was co-administered with anti-PD-1 therapy, pancreatic tumors were further sensitized to the anti-tumor effects of PGEM+CCR2i nanomedicine treatment. That result is significant because clinical trials co-administering anti-PD-1 therapy (or immunotherapy) with traditional chemotherapeutic standard of care to pancreatic cancer patients have largely failed due, in part, to the immunosuppressive tumor microenvironment of pancreatic tumors. The co-administration of PGEM+CCR2i nanomedicine and anti-PD-1 therapy may thus represent an effective treatment strategy for inducing and maintaining an inflammatory tumor microenvironment in “immunologically cold” tumor types.

### Experimental

Materials. Gemcitabine (GEM) was obtained from LC Laboratories (MA, USA). PF-04136309 (PF) and Bindarit (Bind) were purchased from Medchemexpress LLC (USA), BMS813160 (BMS) was purchased from Selleck USA. Vinylbenzyl chloride, potassium carbonate (K₂CO3), azobisisobutyronitrile (AIBN), sodium hydroxide (NaOH), di-tert-butyl decarbonate, 1,4-dioxane, N,N-diisopropylethylamine (DIPEA), triethylamine (TEA), 4-cyano-4-[(dodecylsulfanylthiocarbonyl) sulfanyl] pentanoic acid, dimethylformamide (DMF), tetrahydrofuran (THF), dimethyl sulfoxide (DMSO), N-hydroxy succinimide (NHS), and poly(ethylene glycol) methyl ether methacrylate (OEGMA) were purchased from Fisher Scientific. AIBN was purified by recrystallization in ethanol according to the literature. See Chen, Q., et al., Nature, 533 (7604), 493 (2016). 1-(3-Dimethylaminopropyl)-3-ethylcarbodiimide HCl (EDC-HCL) and 1-hydroxybenzotriazole (HOBT) were purchased from GL Biochem (Shanghai, China). Dulbecco's Modified Eagle's Medium (DMEM) and 3-(4,5-dimethylthiazol-2-yl)-2,5-diphenyl tetrazolium bromide (MTT) was bought from Sigma-Aldrich (MO, USA).

Cells and animals. Murine pancreatic cell line Panc02 was obtained from ATCC (Manassas, VA). KPC cell line was kindly gifted by Professor Wen Xie's lab at University of Pittsburgh. Panc02 was routinely cultured in DMEM supplemented with 10% fetal bovine serum (FBS) and 100 U/mL penicillin/streptomycin at 37° C. in a humidified environment with 5% CO₂. KPC cells were cultured in DMEM (high glucose without sodium pyruvate) with 10% FBS and glutamine (2 mM) as reported.

Female C57/BL6 mice (4-6 weeks old) were purchased from Jackson Laboratory. All animal experiments were conducted following the guidelines of the Institutional Animal Care and Use Committees (IACUC) of University of Pittsburgh.

Preparation and characterization of drug loaded PGEM micelles. PVD polymer was synthesized by controlled Reversible Addition Fragmentation Chain Transfer (RAFT) polymerization. PGEM polymer was synthesized by conjugating GEM to PVD polymer backbone via EDC/HOBT coupling. The polymer structures were characterized by FT-IR.

Blank and CCR2 inhibitors-loaded PGEM micelles were prepared by film hydration method. Briefly, PGEM polymer and CCR2 inhibitors (PF, BMS, and Bind) were mixed in dichloromethane/methanol with various carrier/drug ratios. A thin film was formed after completely removing all the organic solvent via evaporation method. The films were then hydrated with phosphate-buffered saline (PBS) solution to give PF-, BMS-, and Bind-loaded PGEM micelles, respectively. 1,1′-Dioctadecyl-3,3,3′,3′-Tetramethylindotricarbocyanine Iodide (DiR) or rhodamine loaded PGEM micelles and PVD micelles were prepared similarly. Drug loading capacity (DLC) and drug loading efficiency (DLE) were determined by HPLC and calculated according to the following equations:

$$DLC ( \% ) = [ weight of drug loaded / ( weight of polymer + drug used ) ] \times 100 \\ DLE ( \% ) = ( weight of loaded drug / weight of input drug ) \times 100$$

Preparation of three-dimensional tumor spheroids. Panc02 cells and KPC cells were seeded in the Corning 96-well ultra-low-attachment plates with DMEM containing 10% FBS and 6 μg/mL of collagen, respectively. The tumor spheroids were formed after incubation for three days at 37° C. with 5% CO₂. Then, the prepared tumor spheroids were transferred to a new 96-well plate with 100 μL of fresh media containing rhodamine-loaded PGEM and PVD nanoparticles. After incubation for 16 h, the tumor spheroids were washed with cold PBS and the z-stack images were obtained by laser confocal microscopy.

Establishment of orthotopic KPC pancreatic tumor model. Orthotopic KPC xenograft model was established according to previous publication. Briefly, the animals were anesthetized with continuous isoflurane, and the surgical site was sterilized by repetitive application of betadine and 70% ethanol. The surgery was performed in an appropriate position: animals were placed on a water heating pad and the surgical site was draped with sterile gauze. In brief, a surgical incision of 0.5˜0.7 cm was made in the left flank of mice to expose the injection site, followed by an injection of 50 μL of DMEM/Matrigel (1:1, v/v) containing 1×10⁶ KPC cells into the tail of the pancreas. The surgery area was then closed carefully. The mice were kept on the warming pads until full recovery from the anesthesia status and then transferred to clean cages and maintained in pathogen-free condition.

Bulk RNA-Seq Analysis. The Panc02 tumor-bearing mice were intravenously injected with saline, GEM and PGEM once every three days for 3 times, respectively. One day after last injection, the tumors were harvested for RNA sequencing (RNA-seq), which was performed by the Health Sciences Sequencing Core at Children's Hospital at University of Pittsburgh. RNA-Seq libraries were sequenced as 75-bp paired-end reads at a depth of ˜73-77 million reads per sample. Reads were mapped to the mouse genome (GRCm38) using STAR Aligner 2.6.1a. Gene expression quantification and differential expression analysis was performed on Saline, GEM, and PGEM treated tumor samples using RSEM 1.3.1 and Cuffdiff of Cufflinks 2.2.1. Volcano plots were generated to show the overall differential expression between pairwise comparisons among these three groups, where the x-axis indicates the log-transformed fold changes (log 2FC) and the y-axis indicates the corresponding negative log-transformed p-values (−log₁₀(p-value)). The Gene Set Enrichment Analysis (GSEA), via GSEA software available from Broad Institute, Inc. of Cambridge, MA, was further performed based on the gene lists ranked by the log 2FC among three groups.

Western blotting. Western blotting was performed to evaluate the STING activation in cultured Panc02 cells. Cells grown in six-well plates with 80% confluency were treated with saline, free GEM, PGEM micelles, and PVD micelles, respectively, for 48 h. Then, the cultured Panc02 cells were washed twice with pre-cooled PBS and lysed in Pierce™ RIPA buffer (available from Thermo Fischer Scientific of Pittsburgh, PA) for 30 min in 4° C. Protein concentrations were determined by BCA method, and equal amounts of total protein lysate were resolved on a 10% SDS-PAGE and subsequently transferred to a nitrocellulose membrane. Membranes were blocked with 5% non-fat milk in PBS for 1 h prior to incubation with various antibodies targeting IRF3, P-IRF3, STING, P-STING, and Cyclin B dissolved in PBST (DPBS with 0.1% Tween 20) overnight at 4° C. The blots were washed with PBST and then probed with goat antirabbit IgG for 1 h at room temperature followed by chemiluminescence detection by SuperSignal™ West Fento Maximum Sensitivity Substrate (available from Thermo Fischer Scientific). Q-Actin or GAPDH was used as a loading control.

Immunofluorescence Microscopy. Panc02 cells were seeded on chamber slides and treated with saline, free GEM, PGEM micelles, and PVD micelles, respectively. For all immunofluorescence staining, cells were washed in PBS, fixed in 4% formaldehyde/PBS for ten minutes at room temperature (RT) and permeabilized in 0.25% Triton/PBS for 20 minutes at RT. Blocking was performed in 5% BSA/0.1% Triton/PBS for 30 minutes, followed by incubation with primary antibodies in blocking buffer for one hour at 37° C. incubator. Cells were washed in PBS and incubated with secondary antibodies in blocking buffer for one hour at RT, followed by washing and mounting on coverslips using ProLong Gold antifade reagent with DAPI. Cells were imaged using confocal microscope at the specified magnification. For picogreen-cGAS staining, cells were incubated with pico488 DNA quantification reagent for 2 hours at 37° C. For γH2AX staining, permeabilized cells were blocked in Serum-free DAKO Protein Block for one hour at RT. Cells were incubated in primary antibody (γH2AX, 1:500; anti-HA, 1:100) in DAKO antibody (available from Agilent of Santa Clara, CA) diluent at 4° C. overnight on a rocker platform.

qRT-PCR. Panc02 cells grown in six-well plates were treated with saline, free GEM, PGEM micelles, and PVD micelles, respectively, for 48 h. Then, RNA was isolated using the PureLink™ RNA kit (Invitrogen™ available from Thermo Fisher Scientific) and reverse transcribed using the High-Capacity cDNA Reverse Tran--scription Kit (available from Applied Biosystems/Thermo Fisher Scientific) based on the manufacturer's guidelines. qPCR was performed using Power SYBR Green PCR MasterMix (Applied Biosystems) in a QuantStudio 3 Real-Time PCR System (Applied Biosystems).

In a separate study, Panc02 cells grown in six-well plates were treated with PGEM for 72 h. One day after adding PGEM into the wells, control siRNA (siNC) or STING siRNA (siSTING) was added into the wells without changing the medium, and incubated for 48 h. Then the RNA was extracted, and CCL2, CCL7 and ISG15 expression levels were detected as described above. In another study, whether CCL2, CCL7 and ISG15 were induced by type I IFNs was evaluated. Panc02 cells grown in six-well plates were treated with PGEM for 72 h. One day after adding PGEM into the wells, isotype control (10 μg/mL) or IFNAR1 antibody (10 μg/mL) was added into the wells without changing the medium, and incubated for 48 h. Then, the RNA was extracted subjected to qRT-PCR analysis of the mRNA expression levels of CCL2, CCL7 and ISG15.

Molecular simulation systems. A single copy of the PGEM polymer consists of 32 residues from four residue types, which are PG, EM, NPG (N-terminal PG) and CEM (C-terminal EM) (FIG. 10A). Each residue has the hydrophilic and hydrophobic parts. The hydrophilic part tends to be exposed to water, while the hydrophobic part tends to form a hydrophobic core of a micelle structure. For PG and NPG, the hydrophilic part is the polyethylene glycol (PEG) repeat units, and the other atoms belong to the hydrophobic part. For EM and CEM, the hydrophilic part is the substructure containing gemcitabine and the adjacent amide functional group, and the other atoms belong to the hydrophobic part. The four residues and the three drugs were described by the GAFF³¹ force field. See Wang, J., et al., Journal of computational chemistry, 25 (9), 1157 (2004). Specifically, ab initio calculations (B3LYP/6-31G*//HF/6-31G*) using Gaussian 16 package were performed to obtain electrostatic potentials to derive point charges, and then Antechamber software package was applied to generate residue topologies. See, for example, Frisch, M. J., et al., Gaussian 16 Rev. C.01. Wallingford, CT, (2016) and Wang, J., et al., Journal of molecular graphics and modelling, 25 (2), 247 (2006). As there is no experimentally determined structure for PGEM, an initial structure was generated which has an extended conformation. Although the numbers of PG and EM residues are known from experiment, the exact sequence of the polymer is unclear. The PGEM model hereof maximally separates the two types of non-terminal residues and the sequence is as the follows: NPG-EM-EM-EM-PG-EM-EM-PG-EM-EM-EM-PG-EM-EM-PG-EM-EM-EM-PG-EM-EM-PG-EM-EM-EM-PG-EM-EM-PG-EM-EM-CEM.

Molecular dynamics simulations. A series of sequential molecular dynamics (MD) simulations were applied to obtain the micelle structure formed by a single copy of polymer. How long multi-drug molecules interacted with the polymer in explicit water was then studied. In Step 1, the extended conformation of the polymer was collapsed in explicit water during the 100 nanoseconds (ns) simulation. Then a Generalized Born molecular dynamics (GBMD) simulation was performed to exclude the trapped water molecules and accelerate micelle structure formation. The initial conformation of this step came from the last snapshot of Step 1. Next, in Step 3 200-ns MD simulations were performed in explicit water environment to sample an isothermal-isobaric ensemble of the polymer for the followed free energy analysis. Similarly, the initial conformation of this step came from the last MD snapshot of Step 2. Next, in Step 4 220-ns MD simulations for the systems containing a copy of polymer and 8 copies of a drug molecule were carried out. Initially, the 8 copies of drug molecules were placed at the vertices of a cube (edge length=50 Å) with the polymer being placed in the center. The polymer structure was from the last snapshot of Step 3. Finally, for the sake of free energy calculations, a 200-ns M D simulation was performed for a single copy of drug molecule in aqueous solution (Step 5). In total, three drug molecules were studied in this work including Bindarit, BMS813160, and PF-04136309. For both GBMD and explicit water MD simulations, the time step of integrating the Newton's equation of motion was set to 2 femtoseconds, and desired temperature and pressure were set to 298 K and 1 bar, respectively. All MD simulations were performed using the pmemd.cuda program in AMBER 18 software.

Free energy analysis. 500 snapshots from the sampling phase (after 50 ns) of a trajectory were collected for free energy calculations. An internal program was applied to calculate the MM-PBSA-WSAS free energies of the polymer, the drug and the complex formed by the polymer and 8 drug molecules. For a molecular system, the polar solvation free energy was calculated with the Delphi 95 software, using the interior and exterior dielectric constants of 1.0 and 80.0, respectively. The nonpolar solvation free energy was estimated using the solvent accessible surface area (SAS) as detailed elsewhere. The conformational entropy of the system was predicted using WSAS, a weighted solvent-accessible surface area approach. To calculate the binding free energy between the polymer and 8 drug molecules, the “three-trajectories” protocol was adopted, for which the free energies of the polymer, the complex, and the drug were calculated using three MD trajectories separately sampled in Steps 3, 4 and 5. The MM-PBSA-WSAS binding free energy was calculated using the following formula: ΔG_{binding}=G_{complex} −(G_{polymer}+8×G_{drug}). Note that the complex contains 8 copies of the drug molecule.

MTT assay. Panc02 cells were seeded in 96-well plates at a density of 1500 cells/well with 100 μL of complete culture medium (DMEM with 10% FBS and 1% streptomycin/penicillin). After overnight incubation, Panc02 cells were treated with various concentrations of saline, free PF, free BMS, free Bind, free GEM, free GEM+PF, PGEM, PGEM/PF, PGEM/BMS, and PGEM/Bind micelles. After 96 h, the cytotoxicity was determined by MTT assay. The absorbance of each well was measured at 590 nm and the cell viability was determined via the following formula: (OD_{treated}−OD_{blank})/(OD_{control}−OD_{blank})×100%.

Release of PF from PGEM/PF micelles. The release of PF from PGEM/PF micelles was examined at 37° C. via a dialysis method with free PF as a control. PF-loaded PGEM micelles was transferred into a dialysis bag with MWCO of 3500 Da, which was then incubated in 50 mL PBS with 0.5% (w/v) tween 80 under gentle shaking. At specific time intervals, all the PBS solution outside the dialysis bag was withdrawn and replaced with another 50 mL of fresh solution. The PF concentrations in the solution were determined by HPLC.

In vivo anti-tumor study. The antitumor effect was evaluated in syngeneic mice bearing Panc02 tumors. In brief, 1×10⁵ Panc02 cells were inoculated into the right flank of female C57BL/6 mice. When the tumor volume reached ˜50 mm³, the mice started to receive different treatments. To screen the best CCR2-inhibitor loaded PGEM micelles, PF-, Bind-, and BMS-loaded PGEM micelles (10 mg/kg for CCR2 inhibitor) were intravenously administered three times at an interval of three days, while saline and PGEM micelles worked as control groups. In a separate study, the synergistic tumor inhibition effect of PF and GEM was evaluated by intravenously injecting saline, free GEM, free PF, free GEM+PF, PGEM, and PGEM/PF (GEM: 20 mg/kg; PF: 10 mg/kg), respectively, into the Panc02 tumor-bearing mice. These treatments were also performed three times at an interval of three days. The tumor volumes and body weights were continuously monitored. At the endpoint, the tumor tissues and major organs were fixed in 10% formalin and dehydrated. Then they were embedded in paraffin and sectioned, followed by H&E staining with for histopathological examination. The biochemical parameters, including WBCs, RBCs, Hb, AST, ALT, and creatinine, were evaluated for safety profiles. The effect of PGEM/PF on the inhibition of lung metastasis of pancreatic cancer was also evaluated. A pulmonary metastasis model of pancreatic cancer was established by injecting KPC cells via tail vein. On day 10 following tumor cell inoculation, mice were i.v. administered with saline, free GEM+PF, PGEM, and PGEM/PF, respectively. The treatment was performed at day 10, day 14, and Day 18, and the mice were sacrificed at day 20.

In a separate study, the survival of the tumor-bearing mice was examined following various treatments, including saline, free GEM+PF, PGEM, and PGEM/PF (PF dose: 20 mg/kg). The treatments were performed once every 4 days for 3 times. The end point of survival was defined as animal death or when the tumor volume reached ˜2000 mm³. The survival rate was plotted as Kaplan Meier curves.

To test the combination effect of PGEM/PF formulation with anti-PD-1, Panc02 tumor-bearing mice were treated with anti-PD-1 (clone RMPI-14, BioXCell), PGEM/PF or the combination of PGEM/PF formulation and anti-PD-1 (100 μg anti-PD-1 per mouse, i.p. and 5 mg/kg PF, i.v.). The tumor growth was followed every 4 days and tumors were weighed at the completion of the experiment.

Biodistribution and tumor retention of PGEM/PF. The distribution and retention of drug in the tumors was performed in the orthotopic KPC model, which was established according to previous publication. At 3 weeks post inoculation of KPC cells, the mice were intravenously injected with DiR-loaded PGEM micelles at a DiR concentration of 0.06 mg/mL. At 24 h post injection, the tumors and major organs were harvested and the biodistribution of PGEM NPs was evaluated by near infrared fluorescence (NIR) imaging using IVIS 200 system (Perkin Elmer, USA). In a separate study, the tumor retention of PGEM NPs was followed by NIR imaging at indicated time points (0 h, 24 h, 48 h, 72 h, 96 h, 120 h, 144 h, and 168 h).

For the PF quantitation, the tissues were collected and homogenized with PBS. See Sun, J., et al., Acta biomaterialia 106, 289 (2020). The homogenized tissue samples were centrifuged, and 75 μL of the supernatant was transferred to a tube, to which 150 μL methanol containing the internal standard (apixaban-13C-d₃) was added. After protein precipitation, the PF concentration was detected by LC-MS/MS. Chromatographic separation was performed using a Waters Cortecs T3 column (2.1×50 mm, 1.6 μm). An isocratic gradient was used for PF with a total runtime of 6 minutes using the mobile phases water with 0.1% formic acid (A) and acetonitrile (B).

Quantification of tumor-infiltrating lymphocytes by flow cytometry. Syngeneic C57BL/6 mice bearing Panc02 tumors received various treatments (saline, free GEM, free PF, free GEM+PF, PGEM, and PGEM/PF) via i.v. administration every three days for three times. Tumors were collected at 24 h following the last treatment. Single cell suspensions were prepared according to previous literature. See Wan, Z., et al., Science Advances, 7 (50), eabj4226 (2021). Then the cells were stained with various antibodies (Table 1) for flow cytometry analysis. For intracellular cytokine (IFN-γ and GZB) staining, cells were stimulated with PMA (100 ng/mL) and lonomycin (500 ng/mL) for 5 h in the presence of Monensin. Cells were fixed/permeabilized using the BD Cytofix/Cytoperm kit prior to cell staining. Then, the fixed/permeabilized cells were suspended in residual Intracellular Staining Perm Wash Buffer and a predetermined optimum concentration of fluorophore-conjugated antibody of interest was added (e.g. PE anti-IFN-γ) for 30 minutes. The data were analyzed using FlowJo software (available from Becton Dickenson & Company pf Ashland, OR). The percentage of cytokine (IFN-γ or GZB)-producing NK and CD8⁺ T cells was gated under total NK and CD8⁺ T cells, respectively, which represents the percentage of total NK or CD8⁺ T that are IFN-γ or GZB positive.

TABLE 1
Antibodies

| | | |
|---|---|---|
| Anti-mouse CD4S-BUV395 (30-F11) | BD Biosciences | Cat # 564279 |
| Anti-mouse CD45-PerCP (30-F11) | Biolegend | Cat # 103129 |
| Anti BV510 live/dead | Tonbo Biosciences | Cat # 13-0870-T100 |
| Anti-mouse CD8a-BV480 (53-6.7) | BD Biosciences | Cat # 566169 |
| Anti-mouse CD8a-Pacific Blue | BD Biosciences | Cat # 558106 |
| Anti-mouse CD4-BUV737 (GK1.5) | BD Biosciences | Cat # 564298 |
| Anti-mouse CD4-BV785 (GK1.5) | Biolegend | Cat # 100453 |
| Anti-mouse NK1.1-APC (PK136) | BD Biosciences | Cat # 550627 |
| Anti-mouse NK1.1-APC-Cy7 (IgG2a, κ) | BD Biosciences | Cat # 560618 |
| Rat Anti-CD11b-APC (M1/70) | BD Biosciences | Cat # 553312 |
| Anti-mouse Ly-6G/Ly-6C-PE (Gr-1) (RB6-8C5) | BD Biosciences | Cat # 553128 |
| Anti-mouse Ly-60/Ly-6C-BV711 (Gr-1) (RB6-8C5) | Biolegend | Cat # 108443 |
| Anti-mouse I-A/I-E (MHCII)-Pacific Blue (IgG2b) | Biolegend | Cat # 107620 |
| Anti-mouse CD24-BV510 (M1/69) | BD Biosciences | Cat # 747717 |
| Anti-mouse F4/80-APC-Cy7 (BM8) | Biolegend | Cat # 123118 |
| Anti-mouse CD103-PE-CF594 (M290) | BD Biosciences | Cat # 565849 |
| Anti-mouse CD11c-BUV737 (HL3) | BD Biosciences | Cat # 612796 |
| Anti-mouse CD279 (PD-1)-APC-Cy7 (43) | Biolegend | Cat # 135223 |
| Anti-monse CD279 (PD-1)-PE (J43) | BD Biosciences | Cat # 561788 |
| Anti-mouse/human granzyme B-AF647 (GBI1) | Biolegend | Cat # 515405 |
| Anti-mouse/human granzyme B-FITC (GB11) | Biolegend | Cat # 515403 |
| Anti-mouse IFN-r-PE-Cy7 (XMG1.2) | BD Biosciences | Cat # 557649 |
| Anti-mouse Foxp3-PE-CF594 (MF23) | BD Biosciences | Cat # 562466 |
| Anti-mouse Ki-67-Pacific Blue (16A8) | Biolegend | Cat # 652421 |
| Ki-67 mAbs-APC (SolA15) | eBioscience | Cat # 17569880 |
| Phospho-TBK1/NAK (Ser172) (D52C2) XP ® Rabbit mAb | Cell Signaling | Cat # 13498 |
| (PE Conjugate) | Technology | |
| Rabbit (DALE) mAb IgG XP ® Isotype Control (PE | Cell Signaling | Cat # 5742 |
| Conjugate) | Technology | |
| Phospho-IRF-3 (Ser396) (D6O1M) Rabbit mAb (Alexa | Cell Signaling | Cat # 10327 |
| Fluor ® 647 Conjugate) | Technology | |
| Rabbit (DAIE) mAb IgG XP ® Isotype Control (Alexa | Cell Signaling | Cat # 2985 |
| Fluor ® 647 Conjugate) | Technology | |

In a separate study, C57BL/6 mice bearing GFP-Panc02 tumors were treated with saline, GEM and PGEM, respectively, every three days for three times. The tumors were harvested at 24 h following the last treatment, and the single cell suspension was prepared and stained with various antibodies. Compensation was performed using both single color stains and isotype controls, in line with the published protocol. In brief, Phospho-TBK1/NAK (Ser172) (D52C2) XP® Rabbit mAb (PE Conjugate), available from Cell Signaling Technology of Danvers, MA), or concentration matched Rabbit (DA1E) mAb IgG XP® Isotype Control (PE Conjugate) was used for flow cytometric analysis. Similarly, Phospho-IRF-3 (Ser396) (D601M) Rabbit mAb (Alexa Fluor® 647 Conjugate, available from Thermo Fisher Scientific) or concentration matched Rabbit (DA IE) mAb IgG XP® Isotype Control (Alexa Fluor® 647 Conjugate) was used for Flow cytometric analysis.

Neutralization of immune cells and cytokines. To study the contribution of NK and CD8⁺ T cells to the antitumor effect, five mice in each group were intraperitoneally injected with 200 μg of anti-CD8a (clone 2.43, Bio X Cell) or anti-mouse NK1.1 (clone PK136, Bio X Cell) antibody or their matched isotype controls four times (days 6, 9, 12, and 15) after tumor inoculation following previous reported method⁴². In addition, the type I IFNs signaling was blocked using the antibody against the type I interferon receptor (IFNAR-1) to investigate whether the efficacy of PGEM therapy is dependent on type I IFNs. Antibody was administered by intraperitoneal injection on days 6 and 7 at a dose of 200 μg per mouse after tumor inoculation. The tumor volumes and body weights were continuously monitored. At the endpoint, the tumor weights were measured.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A formulation, comprising: a nanostructure comprising an agent to activate a STING pathway in vivo and a C-C chemokine receptor type 2 (CCR2) antagonist.

2. The formulation of claim 1 wherein the nanostructures have a diameter less than 30 nm.

3. The formulation of claim 2 wherein the agent to activate a STING pathway comprises amphiphilic polymers which self-assemble into the nanostructure.

4. The formulation of claim 3 wherein the CCR2 antagonist is physically loaded into the nanostructure.

5. The formulation of claim 4 wherein the amphiphilic polymers comprise one or more molecules that are STING agonists conjugated thereto.

6. The formulation of claim 5 wherein the one or more molecules that are STING agonists are nucleoside analogs.

7. The formulation of claim 6 wherein the nucleoside analog is a cytidine analog which is active as a STING agonist.

8. The formulation of claim 7 wherein the cytidine analog is selected from the group consisting of gemcitabine, azacitidine, decitabine, zebularine, and cytarabine.

9. The formulation of claim 6 wherein the one or more molecules that are STING agonists are attached to the amphiphilic polymer via a linking moiety that is labile in vivo.

10. The formulation of claim 4 wherein the CCR2 antagonist is a small molecule compound.

11. The formulation of claim 10 wherein the CCR2 antagonist has a molecular weight below 1.5 kDa.

12. The formulation of claim 10 wherein the CCR2 antagonist is selected from the group consisting of PF-04136309, PF-04634817, BMS 813160, BMS-687681, BMS-741672, Bindarit, CCX872, CCX140, RDC018, DMX-200, AZD2423, Cenicriviroc, CNTX-6970, JNJ-17166864, MK-0812, RAP-103, RO5234444, SSR150106, WXSH0213, and NOX-E36.

13. The formulation of claim 12 wherein the CCR2 antagonist is PF-04136309.

14. The formulation of claim 12 wherein the one or more molecules that are sting agonists are selected from the group consisting of gemcitabine, azacitidine, decitabine, zebularine, and cytarabine.

15. The formulation of claim 4 wherein the one or more molecules that are sting agonists are gemcitabine.

16. The formulation of claim 4 wherein the amphiphilic polymers comprise a hydrophobic polymer backbone, a plurality of groups being attached to the hydrophobic polymer backbone as pendant groups and which comprise one or more molecules that are STING agonists conjugated thereto, another plurality of pendant groups being attached to the hydrophobic polymer backbone as pendant groups and which comprise at least one hydrophilic polymer.

17. A method of delivering a nanomedicine, comprising: administering a formulation, comprising: a nanostructure comprising an agent to activate a STING pathway in viva and a CCR2 antagonist.

18. The method of claim 17 wherein the nanostructures have a diameter less than 30 nm.

19. The method of claim 18 wherein the agent to activate a STING pathway comprises amphiphilic polymers which self-assemble into the nanostructure.

20. The method of claim 19 wherein the CCR2 antagonist is physically loaded into the nanostructure.

21. The method of claim 20 wherein the amphiphilic polymers comprise one or more molecules that are active as STING agonists conjugated thereto.

22. The method of claim 21 wherein the one or more molecules that are STING agonists are nucleoside analogs.

23. The method of claim 22 further comprising co-delivering an immunotherapy agent with the formulation.

24. The method of claim 23 wherein the immunotherapy agent comprises an anti-programmed death-1 (anti PD-1) composition.

25. The method of claim 24 wherein the anti PD-1 composition comprises a PD-1 antibody.

26. A method of formulating a composition comprising: mixing amphiphilic polymers which self-assemble into nanostructures, wherein the amphiphilic polymers cause activation of a STING pathway in vivo, with a C-C chemokine receptor type 2 (CCR2) antagonist in a liquid medium.
